(19) United States
(12) Patent Application Publication
(10) Pub. No.: US 2024/0307349 A1
(43) Pub. Date: Sep. 19, 2024

(54) METHODS FOR THE PREPARATION AND DOSE REGIMENS FOR USE OF SSTR4 AGONISTS AND SALTS THEREOF

(71) Applicant: Eli Lilly and Company, Indianapolis, IN (US)

(72) Inventors: William Brian KIELBASA, Orchard Park, NY (US); Scott Alan MONK, Indianapolis, IN (US); Eric Michael PEARLMAN, Carmel, IN (US); David Michael REMICK, Fishers, IN (US); John Robert RIZZO, Plainfield, IN (US)

(21) Appl. No.: 18/597,108

(22) Filed: Mar. 6, 2024

Related U.S. Application Data
(60) Provisional application No. 63/489,563, filed on Mar. 10, 2023.

(51) Int. Cl. A61K 31/403; A61K 9/48; A61K 31/4439

(52) U.S. Cl. CPC A61K 31/403; A61K 9/485; A61K 9/4866; A61K 31/4439

(57) ABSTRACT

Method for preparing SSTR4 agonist compounds. Method for preparing SSTR4 agonist compounds through a diasteromerically selective cyclization reaction without the need for a separate epimerization step. Novel intermediates for preparing SSTR4 agonist compounds. Dose regimens for the treatment of pain and/or neuropathic pain, such as diabetic peripheral neuropathy, central neuropathy, or mixed neuropathy.

### FIELD OF THE INVENTION

The present invention is directed to methods for the preparation of SSTR4 agonists. The present invention is also directed methods for the preparation of salts of SSTR4 agonists.

The present invention is also directed to dose regimens for use of SSTR4 agonists, and pharmaceutically acceptable salts and/or hydrates thereof, in the treatment of chronic lower back pain, osteoarthritic pain, and/or neuropathic pain, such as diabetic neuropathy, diabetic peripheral neuropathic pain, polyneuropathy, distal sensory polyneuropathy, peripheral neuropathy, central neuropathy, and/or mixed neuropathy.

### BACKGROUND OF THE INVENTION

Somatostatin, or somatotropin-release inhibitory factor (SRIF), is a cyclic peptide found in humans. It is produced widely in the human body and acts both systemically and locally to inhibit the secretion of various hormones, growth factors and neurotransmitters. The effects of somatostatin are mediated by a family of G protein-coupled receptors, of which five subtypes are known. These subtypes are divided into two subfamilies, the first comprising SSTR2, SSTR3 and SSTR5 and the second SSTR1 and SSTR4.

Somatostatin is involved in the regulation of processes such as for example cellular proliferation, glucose homeostasis, inflammation, and pain. In this aspect, somatostatin or other members of the somatostatin peptide family are believed to inhibit nociceptive and inflammatory processes via the SSTR4 pathway.

WO 2014/184275 discloses certain 3-azabicyclo[3.1.0]hexane-6-carboxamide derivatives which are SSTR4 agonists, and which are useful for preventing or treating medical disorders related to SSTR4. However, it can be challenging to synthesize 3-azabicyclo[3.1.0]hexane-6-carboxamide derivatives with sufficient enantiomeric and diastereomeric purity. Lab scale synthetic pathways are known, but many steps used in previous synthetic pathways can be impractical and/or too expensive to utilize at commercial scale.

Thus, there is a need for alternative ways to prepare certain SSTR4 agonists at commercial scale with sufficient purity. Accordingly, the present invention is directed to methods for the preparation of certain SSTR4 compounds, such as (1R,5S,6r)-N-(2-Methyl-1-((3-methylpyridin-2-yl)oxy)propan-2-yl)-3-azabicyclo[3.1.0]hexane-6-carboxamide and pharmaceutically acceptable salts, solvates, and/or hydrates thereof.

Diabetic neuropathy is a common complication of diabetic microvascular disease. Approximately 40% of patients with diabetes mellitus experience diabetic microvascular disease, and approximately 80% of patients with diabetic neuropathy present with peripheral polyneuropathy.

Symptoms of diabetic peripheral neuropathy include hyperalgesia, paresthesia, and a deep aching sensation. Diabetic peripheral neuropathy (DPNP) symptoms are associated with poor sleep, mobility, depression, and poor quality of life.

Unfortunately, treatment options for DPNP are limited. There are only 3 medications approved by the US FDA for this indication: pregabalin, duloxetine, and tapentadol. While pregabalin and duloxetine each demonstrated efficacy in multiple placebo-controlled clinical trials, both medications require titration to minimize adverse reactions. Tapentadol is an opioid mu-receptor agonist, with the same pharmacological limitations and adverse reaction profile as other opioid analgesics. Other classes of medications are used clinically in an off-label fashion, including gabapentin, selective serotonin norepinephrine uptake inhibitors, tricyclic antidepressants, and anticonvulsants. However, dose-limiting toxicity of all these agents prevents patients from tolerating the therapeutic dose, leading to the use of a subtherapeutic dose in the clinic, and further decreasing the efficacy of these agents. In addition, only 50% of patients continue therapy after 3 months.

Due to the suboptimal dosing and the poor tolerability profile of the nonopioid analgesics used for the management of DPNP, opioids are used as a last resort. Although opioids are efficacious against acute pain, there is evidence suggesting they provide little clinical benefit in chronic pain, not to mention the possibility of reduced efficacy due to tolerance

Additionally, while opioid compounds are known for providing relief from pain symptoms, opioid compounds are also associated with numerous undesirable side effects, including hallucinations, nausea, dizziness, sedation, constipation, urinary retention, dependency, and addiction. As many as 25% of patients undergoing opioid pain therapy for even a short duration can develop a dependence on opioid compounds. In fact, opioid addiction was declared as a National Public Health Emergency on Oct. 26, 2017 by U.S. President Donald Trump. As such, there is a long-felt, but unmet need for the development of non-opioid compounds and dosing regimens for non-opioid compounds to provide relief from pain symptoms without the possibility for the development of addiction and/or dependency.

Accordingly, the present invention is directed to dosing regimens for the treatment of pain, such as neuropathic pain and/or diabetic neuropathy and/or mixed neuropathy, with non-opioid compounds.

While methods for the preparation of certain SSTR4 compounds are disclosed in WO 2014/184275, WO 2021/233427, and WO 2022/012534, these compounds were produced at laboratory scale, which can include synthetic steps that are impractical at commercial scale. In these previous preparations, the synthetic pathways used: (1) expensive catalysts, including palladium and/or rhodium, and/or used lithium aluminum hydride to perform reduction steps, and (2) led to a mixture of diastereomers that needed epimerization after the cyclization of the heterocycle. Both (1) and (2) can increase the overall cost of the preparation and add additional purification steps to remove trace amounts of Pd, Rh, Li, Al, and/or undesirable diastereomers with lower activity.

Disclosed herein, is a new route to certain SSTR4 compounds, such as (1R,5S,6r)-N-(2-Methyl-1-((3-methylpyridin-2-yl)oxy)propan-2-yl)-3-azabicyclo[3.1.0]hexane-6-carboxamide and salts thereof, which can be performed at commercial scale without the use of transition metal catalysts, such as Pd, Pt, Mo, Rh, Fe, Ni, Cr, W, or combinations thereof, and/or aluminum hydride salts, such as lithium aluminum hydride. Additionally, the disclosed method includes a cyclization step including a treatment with a (2-haloethyl)diphenylsulfonium salt which leads to a single diastereomer intermediate without requiring a subsequent epimerization step. Finally, the disclosed method has fewer total steps than the previously disclosed routes, such as less than 8 or from 4 to 7 total steps.

Also disclosed herein is a method of treating pain in a patient in need thereof, through the administration of (1R,5S,6r)-N-(2-methyl-1-((3-methylpyridin-2-yl)oxy)propan-2-yl)-3-azabicyclo[3.1.0]hexane-6-carboxamide, and pharmaceutically acceptable salts and/or hydrates thereof, such as (1R,5S,6r)-N-(2-methyl-1-((3-methylpyridin-2-yl)oxy)propan-2-yl)-3-azabicyclo[3.1.0]hexane-6-carboxamide L-tartrate sesquihydrate. Also disclosed herein is a method of treating chronic lower back pain, osteoarthritic pain, and/or neuropathic pain, such as diabetic neuropathy, diabetic peripheral neuropathic pain, polyneuropathy, distal sensory polyneuropathy, peripheral neuropathy, central neuropathy, and/or mixed neuropathy, to a patient in need thereof, through the administration of (1R,5S,6r)-N-(2-methyl-1-((3-methylpyridin-2-yl)oxy)propan-2-yl)-3-azabicyclo[3.1.0]hexane-6-carboxamide, and pharmaceutically acceptable salts and/or hydrates thereof, such as (1R,5S,6r)-N-(2-methyl-1-((3-methylpyridin-2-yl)oxy)propan-2-yl)-3-azabicyclo[3.1.0]hexane-6-carboxamide L-tartrate sesquihydrate.

(1R,5S,6r)-N-(2-methyl-1-((3-methylpyridin-2-yl)oxy)propan-2-yl)-3-azabicyclo[3.1.0]hexane-6-carboxamide, a non-opioid compound, is a SSTR4 agonist compound. (1R,5S,6r)-N-(2-methyl-1-((3-methylpyridin-2-yl)oxy)propan-2-yl)-3-azabicyclo[3.1.0]hexane-6-carboxamide, and pharmaceutically acceptable salts and/or hydrates thereof, such as (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, are a potential first in class treatment for certain pain conditions, such as chronic lower back pain, osteoarthritic pain, and/or neuropathic pain, such as diabetic neuropathy, diabetic peripheral neuropathic pain, polyneuropathy, distal sensory polyneuropathy, peripheral neuropathy, central neuropathy, and/or mixed neuropathy. Accordingly, disclosed herein, are dosing regimens with demonstrated clinical efficacy for the treatment of certain pain conditions, such as chronic lower back pain, osteoarthritic pain, and/or neuropathic pain, such as diabetic neuropathy, diabetic peripheral neuropathic pain, polyneuropathy, distal sensory polyneuropathy, peripheral neuropathy, central neuropathy, and/or mixed neuropathy.

### BRIEF DESCRIPTION OF THE FIGURE

FIG. 1 shows the mean change of Average Pain Intensity in a Phase 2 study for the treatment of diabetic peripheral neuropathy with (1R,5S,6r)-N-(2-methyl-1-((3-methylpyridin-2-yl)oxy)propan-2-yl)-3-azabicyclo[3.1.0]hexane-6-carboxamide L-tartrate sesquihydrate

### SUMMARY OF THE INVENTION

Disclosed herein are dose regimens for use of SSTR4 agonist compounds, and pharmaceutically acceptable salts and/or hydrates thereof, in the treatment of chronic lower back pain, osteoarthritic pain, and/or neuropathic pain, such as diabetic neuropathy, diabetic peripheral neuropathic pain, polyneuropathy, distal sensory polyneuropathy, peripheral neuropathy, central neuropathy, and/or mixed neuropathy.

Also disclosed herein is a capsule composition comprising certain SSTR4 agonist compounds, and pharmaceutically acceptable salts and/or hydrates thereof, such as (1R,5S,6r)-N-(2-methyl-1-((3-methylpyridin-2-yl)oxy)propan-2-yl)-3-azabicyclo[3.1.0]hexane-6-carboxamide L-tartrate sesquihydrate.

Also disclosed herein is a method for the preparation of certain SSTR4 agonist compounds, and pharmaceutically acceptable salts and/or hydrates thereof, such as 1R,5S,6r)-N-(2-methyl-1-((3-methylpyridin-2-yl)oxy)propan-2-yl)-3-azabicyclo[3.1.0]hexane-6-carboxamide.

Also disclosed herein are dose regimens for the use of (1R,5S,6r)-N-(2-methyl-1-((3-methylpyridin-2-yl)oxy)propan-2-yl)-3-azabicyclo[3.1.0]hexane-6-carboxamide L-tartrate sesquihydrate in the treatment of neuropathic pain, such as diabetic neuropathy, diabetic peripheral neuropathic pain, polyneuropathy, distal sensory polyneuropathy, peripheral neuropathy, central neuropathy, and/or mixed neuropathy.

Also disclosed herein is a method of treating pain in a patient in need of treatment thereof, the method comprising administering to the patient a dose of about 25 mg to about 1400 mg per dose of a compound of the formula:

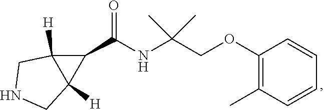 [SMILES: Cc1ccccc1OCC(C)(C)NC(=O)[C@H]1[C@@H]2CNC[C@@H]21]

or a pharmaceutically acceptable salt thereof, and/or a hydrate thereof.

Also disclosed herein is a method of treating pain in a patient in need of treatment thereof, the method comprising administering to the patient a dose of about 25 mg to about 1400 mg per dose of a compound of the formula:

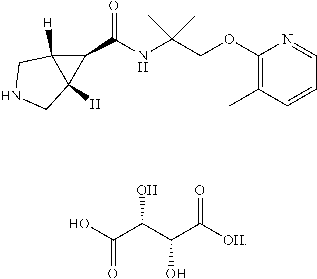 [SMILES: Cc1cccnc1OCC(C)(C)NC(=O)[C@H]1[C@@H]2CNC[C@@H]21 ; O=C(O)[C@H](O)[C@@H](O)C(=O)O]

Also disclosed herein is a method of treating neuropathic pain in a patient in need of treatment thereof, the method comprising administering to the patient a dose of about 50 mg to about 600 mg per dose of a compound of the formula:

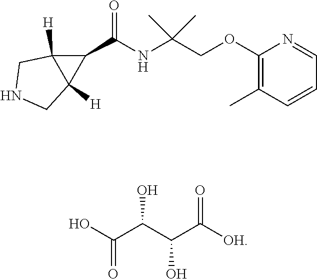 [SMILES: Cc1cccnc1OCC(C)(C)NC(=O)[C@H]1[C@@H]2CNC[C@@H]21 ; O=C(O)[C@H](O)[C@@H](O)C(=O)O]

Also disclosed herein is a method of treating pain in a patient in need of treatment thereof, the method comprising administering to the patient a dose of about 25 mg to about 1400 mg per dose of a compound of the formula:

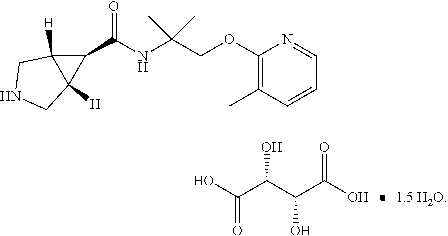

Also disclosed herein is a method of treating neuropathic pain in a patient in need of treatment thereof, the method comprising administering to the patient a dose of about 50 mg to about 600 mg per dose of a compound of the formula:

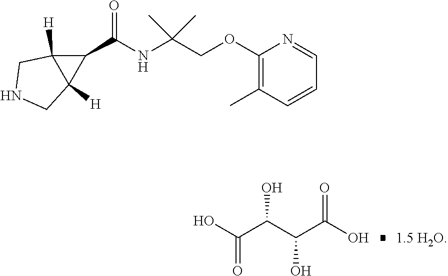 [SMILES: Cc1cccnc1OCC(C)(C)NC(=O)[C@H]1[C@@H]2CNC[C@@H]21 ; O=C(O)[C@H](O)[C@@H](O)C(=O)O]

Also disclosed herein is a novel method for the preparation of SSTR4 agonist compounds, and pharmaceutically acceptable salts thereof and/or hydrates thereof, at commercial scale.

Also disclosed herein is a capsule composition comprising a compound of the formula:

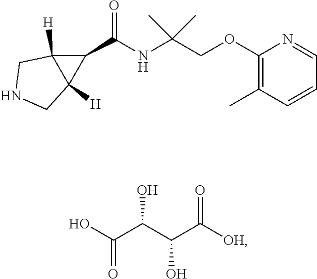 [SMILES: Cc1cccnc1OCC(C)(C)NC(=O)[C@H]1[C@@H]2CNC[C@@H]21 ; O=C(O)[C@H](O)[C@@H](O)C(=O)O]

or a hydrate thereof. Also disclosed herein is a method for preparing a compound of the formula:

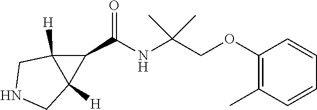 [SMILES: Cc1ccccc1OCC(C)(C)NC(=O)[C@H]1[C@@H]2CNC[C@@H]21]

or pharmaceutically acceptable salts thereof, and the method comprising: mixing a compound of the formula:

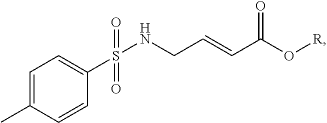

wherein R is a C₁ to C₆ alkyl,
with a sulfonium salt of the formula:

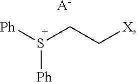

wherein X is a halogen and A is an anion to yield an intermediate compound of the formula:

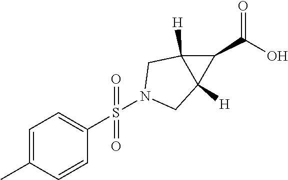 [SMILES: Cc1ccc(S(=O)(=O)N2C[C@@H]3[C@H](C2)[C@H]3C(=O)O)cc1]

mixing the intermediate compound with

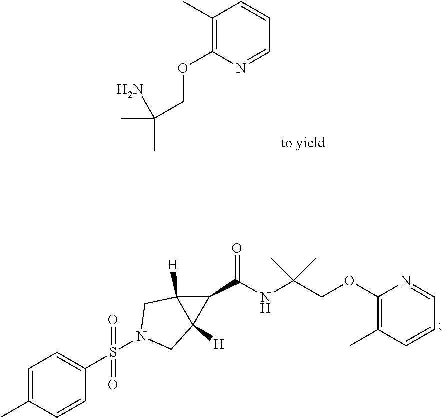 [SMILES: Cc1cccnc1OCC(C)(C)N ; Cc1ccc(S(=O)(=O)N2C[C@@H]3[C@H](C2)[C@H]3C(=O)NC(C)(C)COc2ncccc2C)cc1]

and

removing tosyl function group from

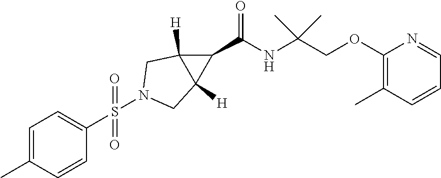 [SMILES: Cc1ccc(S(=O)(=O)N2C[C@@H]3[C@H](C2)[C@H]3C(=O)NC(C)(C)COc2ncccc2C)cc1]

to yield the compound.

Also disclosed herein is a product comprising a compound, such as (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, prepared by the method described herein.

### DETAILED DESCRIPTION OF THE INVENTION

### SSTR4 Agonists

The disclosed method can be used to prepare the compounds of Formula I. The disclosed method can also be used to prepare salts, solvates, hydrates, and/or combinations thereof of Formula I.

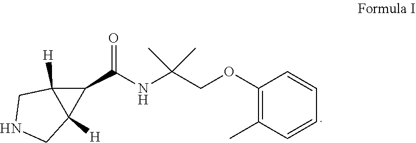 [SMILES: Cc1ccccc1OCC(C)(C)NC(=O)[C@H]1[C@@H]2CNC[C@@H]21]

### SSTR4 Agonist, (1R,5S,6r)-N-(2-methyl-1-((3-methylpyridin-2-yl)oxy)propan-2-yl)-3-azabicyclo[3.1.0]hexane-6-carboxamide

The disclosed method can be used to prepare the compounds of Formula II. The disclosed method can also be used to prepare solvates, hydrates, and/or combinations thereof of Formula II.

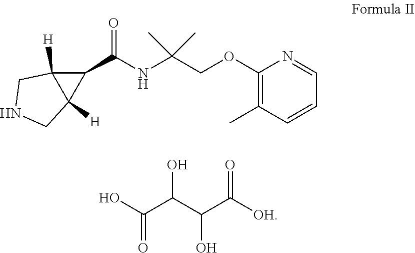

### Tartrate Salt of SSTR4 Agonist, (1R,5S,6r)-N-(2-methyl-1-(3-methylpyridin-2-yl)oxy)propan-2-yl)-3-azabicyclo[3.1.0]hexane-6-carboxamide tartrate

The disclosed method can be used to prepare the compounds of Formula III. The disclosed method can also be used to prepare solvates, hydrates, and/or combinations thereof of Formula III.

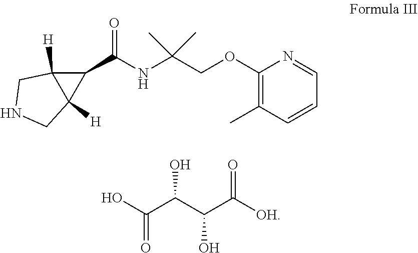

### L-Tartrate Salt of SSTR4 Agonist, (1R,5S,6r)-N-(2-methyl-1-((3-methylpyridin-2-yl)oxy)propan-2-yl)-3-azabicyclo[3.1.0]hexane-6-carboxamide L-tartrate

The disclosed method can be used to prepare the compound of Formula IV.

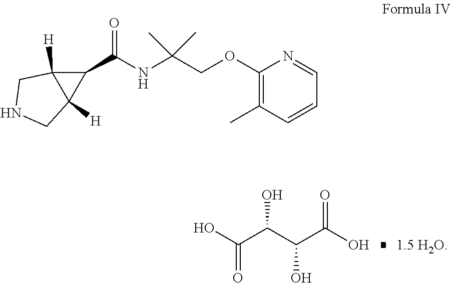 [SMILES: Cc1cccnc1OCC(C)(C)NC(=O)[C@H]1[C@@H]2CNC[C@@H]21 ; O=C(O)[C@H](O)[C@@H](O)C(=O)O]

### (1R,5S,6r)-N-(2-methyl-1-((3-methylpyridin-2-yl)oxy)propan-2-yl)-3-azabicyclo[3.1.0]hexane-6-carboxamide L-tartrate sesquihydrate

### Method

Disclosed herein is a method for preparing a SSTR4 agonist compound. The method for preparing a SSTR4 agonist compound can be used in the commercial scale production of an SSTR4 agonist compound, a pharmaceutically acceptable salt, and/or a solvate/hydrate thereof, as further discussed herein. In the disclosed method, preferably the reactions are performed using batch processing methodology. In an embodiment, the batches are produced at process scale. In an embodiment the batches are produced in at least 1 kilogram. In an embodiment, the batches by are produced in at least 10 kilograms. In an embodiment, the batches are produced in at least 100 kilograms.

The method for preparing a SSTR4 agonist compound of the formula:

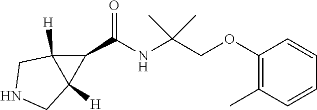 [SMILES: Cc1ccccc1OCC(C)(C)NC(=O)[C@H]1[C@@H]2CNC[C@@H]21]

or a pharmaceutically acceptable salts, solvates, or hydrates thereof, can comprises:

### (a) Mixing tert-Butyl tosyl carbamate with a suitable C₁-C₆ Alkyl 4-halobut-2-enoate

Step (a) of the method to prepare a SSTR4 agonist compound can include the mixing tert-Butyl tosyl carbamate with a suitable C₁-C₆ Alkyl 4-halobut-2-enoate compound in a polar aprotic solvent as shown in Formula V, wherein X is a halogen leaving group, such as Cl, Br, or I.

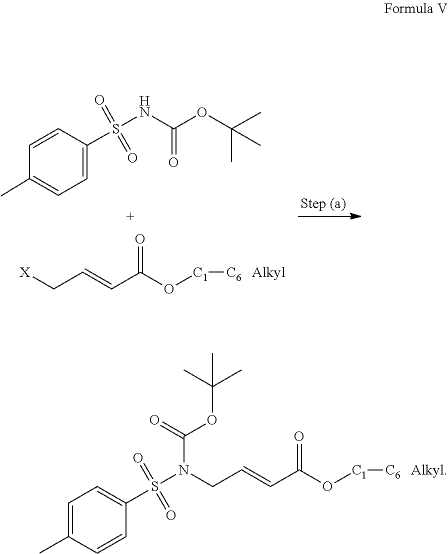

### Step (a) of Method for Preparing SSTR4 Agonist Compound

The components of step (a) can be combined in a polar aprotic solvent at a temperature of from about 10° C. to about 25° C., from about 15° C. to 25° C., or from 20° C. to 25° C. The temperature of the mixture of components in step (a) can be increased to from about 25° C. to about 50° C., from about 25° C. to about 40° C., or about 30° C. The product mixture of step (a) can be filtered, and the collected residue washed with a polar aprotic solvent to yield the product in solution.

Suitable polar aprotic solvents are well known to a person of ordinary skill in the art of organic synthesis design. Suitable polar aprotic solvents can include acetonitrile, acetone, dimethyl sulfoxide, N,N-dimethylformamide, tetrahydrofuran, among others.

In another embodiment, the method can include the mixing tert-Butyl tosyl carbamate with (E) Methyl 4-bromobut-2-enoate compound in acetonitrile to yield Methyl (E)-4-((N-(tert-butoxycarbonyl)-4-methylphenyl)sulfonamido)but-2-enoate, as shown in Formula VI.

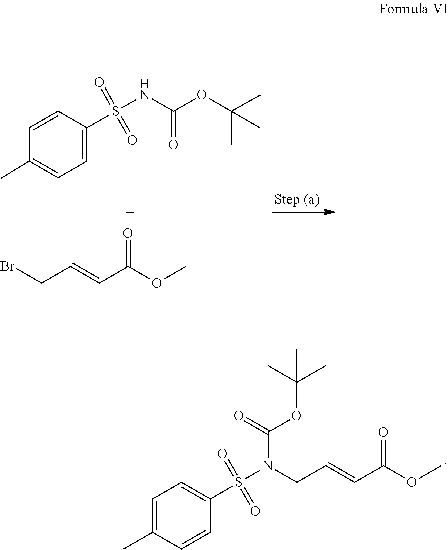

### Step (a) of Method for Preparing SSTR4 Agonist Compound

In another embodiment, step (a) can comprise mixing

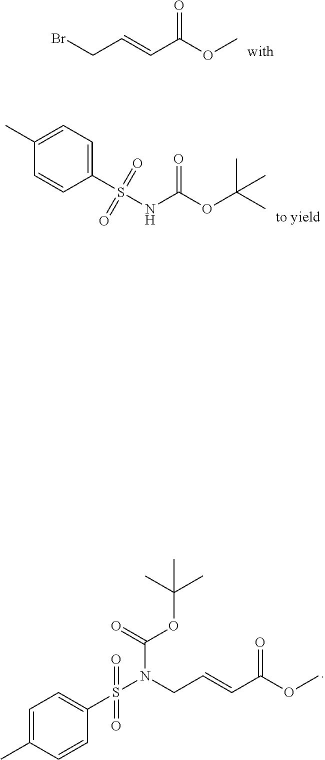

Additional components to step (a) may include a base, such as potassium carbonate, and/or a salt, such as potassium iodide.

### (b) Mixing C₁-C₆ Alkyl (E)-4-((N-(tert-butoxycarbonyl)-4-methylphenyl)sulfonamido)but-2-enoate with an acid

Step (b) of the method to prepare a SSTR4 agonist compound can include the mixing the product generated in step (a), C₁-C₆ Alkyl (E)-4-((N-(tert-butoxycarbonyl)-4-methylphenyl)sulfonamido)but-2-enoate, with a suitable acid in a polar aprotic solvent as shown in Formula VII.

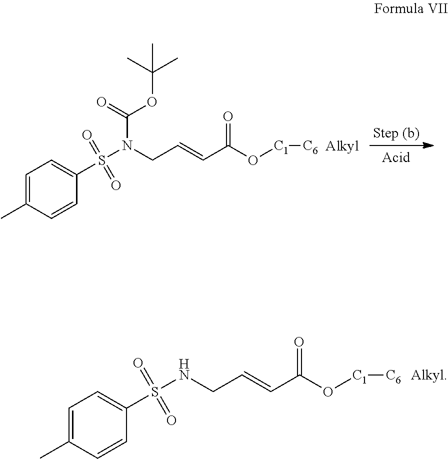

### Step (b) of Method for Preparing SSTR4 Agonist Compound

The components of step (b) can be combined in a polar aprotic solvent at a temperature of from about 10° C. to about 25° C., from about 15° C. to 25° C., or from 20° C. to 25° C. The temperature of the mixture of components in step (b) can be increased to from about 40° C. to about 75° C., from about 40° C. to about 60° C., or from about 55° C. to about 60° C. The reaction solution can remain at the elevated temperature for at least 4 hours, at least 10 hours, or at least 12 hours.

After mixture of the step (b) components are heated to the elevated temperatures, the solution can be concentrated and the remaining polar aprotic solvent exchanged with toluene and/or ethyl acetate.

The polar aprotic solvent can be the same solvent as in step (a) so that the product can remain in solution without any additional purification steps other than filtration of byproducts or the solvent can be a different polar aprotic solvent.

The suitable acid in step (b) can be any acid that can be used to remove the tert-butyl ester from the tertiary amine to form a secondary amine. Suitable acids include trifluoroacetic acid, hydrochloric acid, sulfuric acid, hydrofluoric acid, among other acids.

In another embodiment, step (b) can comprise mixing methyl (E)-4-((N-(tert-butoxycarbonyl)-4-methylphenyl)sulfonamido)but-2-enoate with trifluoroacetic acid in acetonitrile to yield methyl (E)-4-((4-methylphenyl)sulfonamido)but-2-enoate as shown in Formula VIII.

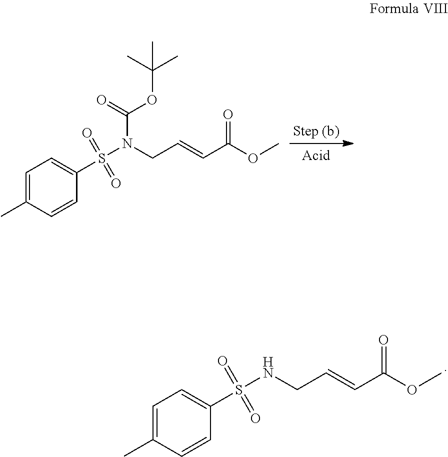

### Step (b) of Method for Preparing SSTR4 Agonist

In another embodiment, step (b) can comprise mixing

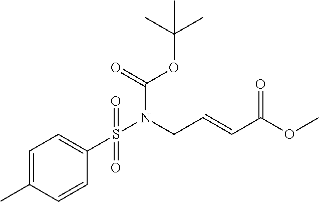

with trifluoroacetic acid to yield

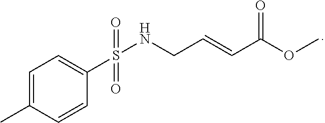

### Step (c) Mixing C₁-C₆ Alkyl (E)-4-((4-methylphenyl)sulfonamido)but-2-enoate with Sulfonium Salt

Step (c) of the method to prepare a SSTR4 agonist compound can include the mixing the product generated in step (b), C₁-C₆ Alkyl (E)-4-((4-methylphenyl)sulfonamido)but-2-enoate, with a sulfonium salt in a suitable heterocycle solvent as shown in Formula IX to yield an intermediate compound, (1R,5S)-3-(p-Tolylsulfonyl)-3-azabicyclo[3.1.0]hexane-6-carboxylic acid.

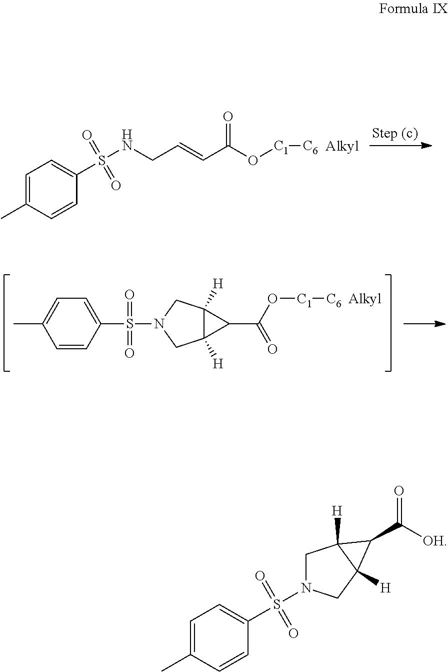

### Step (c) of Method for Preparing SSTR4 Agonist

The components of step (c) can be combined at from −10° C. to 10° C. or about 0° C. In addition to the sulfonium salt and the C₁-C₆ Alkyl (E)-4-((4-methylphenyl)sulfonamido)but-2-enoate, potassium fluoride and/or potassium hydroxide may be added. The temperature of the solution can be gradually or step-wise increased to 30° C. over a period of from about 10 hours to about 30 hours.

Suitable heterocycle solvents include tetrahydrofuran, furan, 2-methyltetrahydrofuran, among others.

After the temperature of the solution has been increased, a base, such as lithium hydroxide, can be added at the increased temperature and the reaction can be allowed to proceed for an additional period of time. The additional period of time can be from about from 8 hours to about 24 hours, from about 12 hours to about 18 hours, or about 16 hours.

The intermediate compound, (1R,5S)-3-(p-Tolylsulfonyl)-3-azabicyclo[3.1.0]hexane-6-carboxylic acid can be separated from solution using traditional synthetic organic chemistry separation techniques well known to a person of ordinary skill in the art.

The sulfonium salt used in step (c) can be represented by Formula X, wherein X is a halogen leaving group, such as Cl, Br, or I and A is any suitable anion, which will be well known to a person of ordinary skill in the art.

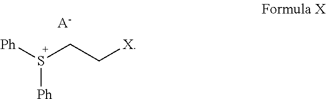

### Sulfonium Salt for Step (c)

The sulfonium salt used in step (c) can also be represented by Formula XI.

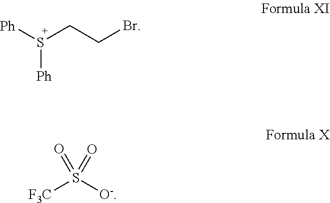

### Sulfonium Salt for Step (c)

In another embodiment, step (c) can comprise mixing methyl (E)-4-((4-methylphenyl)sulfonamido)but-2-enoate with 2-(bromoethyl)diphenylsulfonium triflate to yield (1R,5S)-3-(p-Tolylsulfonyl)-3-azabicyclo[3.1.0]hexane-6-carboxylic acid, the intermediate compound as shown in Formula XII.

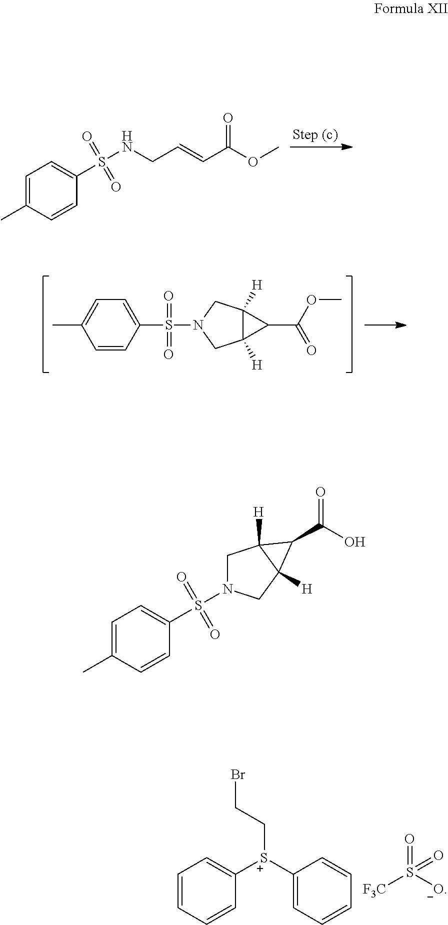

### Step (c) of Method for Preparing SSTR4 Agonist

In another embodiment, step (c) can comprise mixing

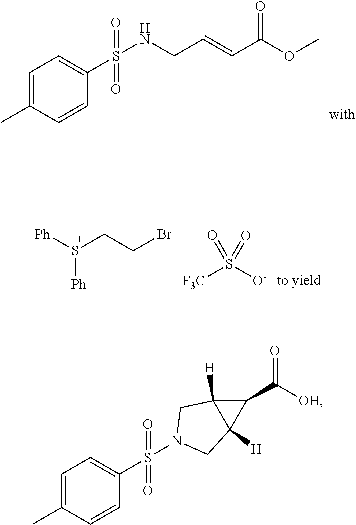

the intermediate compound, or salts or solvates thereof.

### Step (d) Mixing (1R,5S)-3-(p-Tolylsulfonyl)-3-azabicyclo[3.1.0]hexane-6-carboxylic acid with 2-Methyl-1-((3-methylpyridin-2-yl)oxy)propan-2-amine

Step (d) of the method to prepare a SSTR4 agonist compound can include the mixing the intermediate compound generated in step (c), (1R,5S)-3-(p-Tolylsulfonyl)-3-azabicyclo[3.1.0]hexane-6-carboxylic acid, with 2-Methyl-1-((3-methylpyridin-2-yl)oxy)propan-2-amine as shown in Formula XIII.

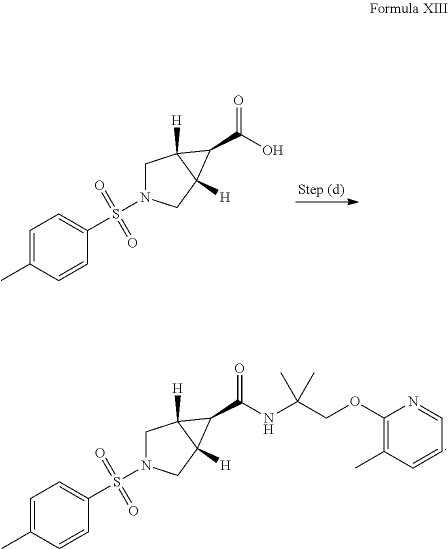 [SMILES: Cc1ccc(S(=O)(=O)N2C[C@@H]3[C@H](C2)[C@H]3C(=O)O)cc1 ; Cc1ccc(S(=O)(=O)N2C[C@@H]3[C@H](C2)[C@H]3C(=O)NC(C)(C)COc2ncccc2C)cc1]

### Step (d of Method for Preparing SSTR4 Agonist

Prior to the addition of 2-Methyl-1-((3-methylpyridin-2-yl)oxy)propan-2-amine, the intermediate compound can be reacted with a suitable reagent, such as oxalyl chloride or thionyl chloride, to generate an acid chloride in situ, replacing the —OH functional group with a —Cl under synthetic conditions well known to a person of ordinary skill in the art.

The generated acid chloride can be combined with 2-Methyl-1-((3-methylpyridin-2-yl)oxy)propan-2-amine at a temperature of from about −10° C. to about 10° C. or from about 0° C. to about 10° C.

The solution temperature can be increased to from about 15° C. to about 25° C. after from about 2 to about 4 hours.

In another embodiment, step (d) can comprise mixing intermediate compound with

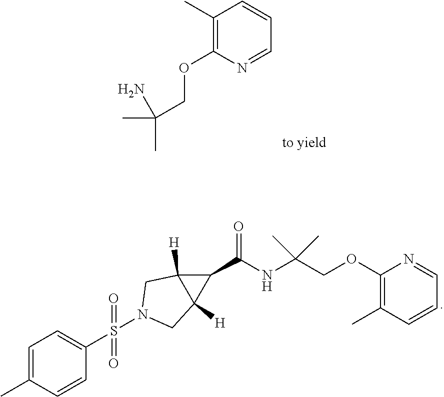 [SMILES: Cc1cccnc1OCC(C)(C)N ; Cc1ccc(S(=O)(=O)N2C[C@@H]3[C@H](C2)[C@H]3C(=O)NC(C)(C)COc2ncccc2C)cc1]

### Step (e) Mixing ((1R,5S,6r)-N-(2-Methyl-1-((3-methylpyridin-2-yl)oxy)propan-2-yl)-3-tosyl-3-azabicyclo[3.1.0]hexane-6-carboxamide with potassium diphenylphosphine

Step (e) of the method to prepare a SSTR4 agonist compound can include the mixing the product generated in step (d), ((1R,5S,6r)-N-(2-Methyl-1-((3-methylpyridin-2-yl)oxy)propan-2-yl)-3-tosyl-3-azabicyclo[3.1.0]hexane-6-carboxamide, with potassium diphenylphosphine as shown in Formula XIV.

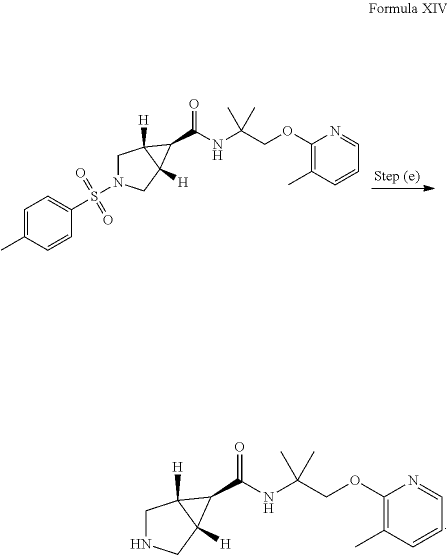 [SMILES: Cc1ccc(S(=O)(=O)N2C[C@@H]3[C@H](C2)[C@H]3C(=O)NC(C)(C)COc2ncccc2C)cc1 ; Cc1cccnc1OCC(C)(C)NC(=O)[C@H]1[C@@H]2CNC[C@@H]21]

### Step (e) of Method for Preparing SSTR4 Agonist

((1R,5S,6r)-N-(2-Methyl-1-((3-methylpyridin-2-yl)oxy)propan-2-yl)-3-tosyl-3-azabicyclo[3.1.0]hexane-6-carboxamide can be dissolved in a suitable solvent, such as methyl tert-butyl ether. The temperature can be decreased to from about −100° C. to about −50° C., from about −80° C. to about −55° C., or from about −70° C. to about −60° C. Potassium diphenylphosphine can be added dropwise with the decreased temperature maintained.

After the entire amount of potassium diphenylphosphine has been added, the solution decreased temperature can be maintained for at least 4 hours, at least 6 hours, or at least 8 hours. The solution can then be allowed to rise to a temperature of from about 15° C. to about 25° C. and the SSTR agonist compound, (1S,5R)-N-[1,1-Dimethyl-2-[(3-methyl-2-pyridyl)oxy]ethyl]-3-azabicyclo[3.1.0]hexane-6-carboxamide can be isolated from solution using typical methods well known to a person of ordinary skill in the art.

In another embodiment, step (e) can comprise mixing

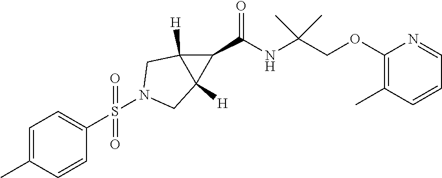 [SMILES: Cc1ccc(S(=O)(=O)N2C[C@@H]3[C@H](C2)[C@H]3C(=O)NC(C)(C)COc2ncccc2C)cc1]

with potassium diphenylphosphine to yield the SSTR4 agonist compound.

### Salts of SSTR4 Agonist Compound

Also disclosed herein is a method for preparing a pharmaceutically acceptable salt of a SSTR4 agonist compound. The method for preparing a pharmaceutically acceptable salt of a SSTR4 agonist compound can comprise (1) the disclosed method for preparing a SSTR4 agonist compound and (2) generating a pharmaceutically acceptable salt of the SSTR4 agonist through: (i) reaction with an acid, (ii) a salt metathesis reaction, and/or (iii) other reactions that can result in the formation of a pharmaceutically acceptable salt of the SSTR4 agonist compound.

Suitable pharmaceutically acceptable salts of SSTR4 agonist compounds can comprise adipate, bromide, chloride, besylate, esylate, mesylate, tosylate, phosphate, succinate, sulfate, citrate, tartrate, L-tartrate, malate, and/or L-malate anions. Other suitable salts can include (1S,5R,6r)-N-[1,1-Dimethyl-2-[(3-methyl-2-pyridyl)oxy]ethyl]-3-azabicyclo[3.1.0]hexane-6-carboxamide tartrate, (1S,5R,6r)-N-[1,1-Dimethyl-2-[(3-methyl-2-pyridyl)oxy]ethyl]-3-azabicyclo[3.1.0]hexane-6-carboxamide L-tartrate, and/or (1R,5S,6r)-N-(2-methyl-1-((3-methylpyridin-2-yl)oxy)propan-2-yl)-3-azabicyclo[3.1.0]hexane-6-carboxamide L-tartrate sesquihydrate. Other suitable salts are shown in Formula II-IV. L-tartrate can also be known as (2R, 3R)-2,3-dihydroxysuccinate.

### Intermediate Compound

Also disclosed herein are new intermediate compounds generated during the disclosed method for the preparation of the SSTR4 agonist compounds or pharmaceutically acceptable salts thereof, hydrates thereof, and/or solvates thereof. Suitable intermediates can be generated at any time during the disclosed method. Suitable intermediates can be isolated as a neat compound or generated only in solution.

Suitable intermediates for the preparation of SSTR4 agonist compounds or pharmaceutically acceptable salts thereof, hydrates thereof, and/or solvates thereof, can include the intermediates of Formula XV or pharmaceutically acceptable salts thereof, hydrates thereof, and/or solvates thereof, wherein Y is H, OH, NH₂ Cl, Br, I, C₁ to C₆ alkyl, C₁ to C₆ ether, or combinations thereof, R₁ and R₂ are independently H, OH, NH₂ Cl, Br, I, C₁ to C₆ alkyl, C₁ to C₆ ether, or combinations thereof, and the phenyl functional group is substituted with one or more R′ and wherein each R′ group are independently H, OH, NH₂ Cl, Br, I, C₁ to C₆ alkyl, C₁ to C₆ ether, or combinations thereof. In an embodiment, Y can be OH, Cl, Br, I, or NH₂ R₁ and R₂ can be H, and R′ can be CH₃ in the para position.

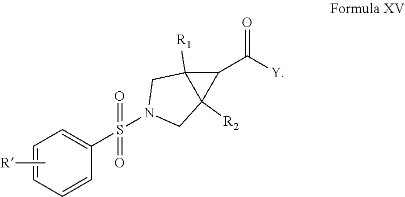

### Intermediate Compound

In an embodiment, the intermediate compound can also be represented by Formula XVI or pharmaceutically acceptable salts thereof, hydrates thereof, and/or solvates thereof, wherein Y can be H, OH, NH₂ Cl, Br, I, C₁ to C₆ alkyl, C₁ to C₆ ether, or combinations thereof, R₁ and R₂ independently can be H, OH, NH₂ Cl, Br, I, C₁ to C₆ alkyl, C₁ to C₆ ether, or combinations thereof; and the phenyl functional group is substituted with one or more R′ and wherein each R′ group are independently H, OH, NH₂ Cl, Br, I, C₁ to C₆ alkyl, C₁ to C₆ ether, or combinations thereof. In an embodiment, Y can be OH, Cl, Br, I, or NH₂ R₁ and R₂ can be H, and R′ can be CH₃ in the para position.

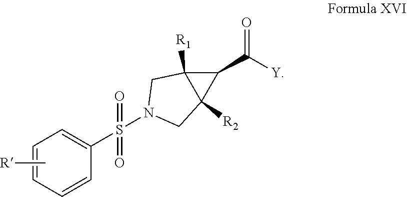

### Intermediate Compound

In an embodiment, the intermediate compound can also be represented by Formula XVII or pharmaceutically acceptable salts thereof, hydrates thereof, and/or solvates thereof, Y can be H, OH, NH₂ Cl, Br, I, C₁ to C₆ alkyl, C₁ to C₆ ether, or combinations thereof. In an embodiment, Y can be OH, NH₂ Cl, Br, or I.

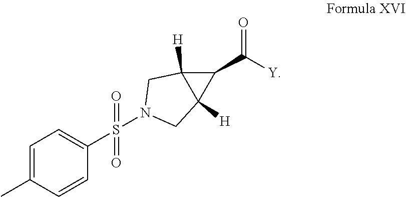

### Intermediate Compound

In an embodiment, the intermediate compound can also be represented by Formula XVIII or pharmaceutically acceptable salts thereof, hydrates thereof, and/or solvates thereof.

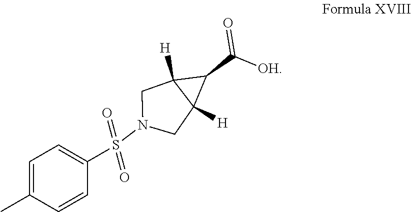 [SMILES: Cc1ccc(S(=O)(=O)N2C[C@@H]3[C@H](C2)[C@H]3C(=O)O)cc1]

### Intermediate Compound

The intermediate compound, as described herein, can be used for the preparation of the SSTR4 agonist compound or pharmaceutically acceptable salts thereof, hydrates thereof, and/or solvates thereof. The intermediate compound offers a stable scaffold to produce a variety of SSTR4 agonist compounds through an amide-carboxylic acid coupling reaction, such as in step (d) of the method for preparing the SSTR4 agonist compound, as described herein and illustrated in Formula VIII.

### Compositions

Also disclosed herein is a pharmaceutical composition comprising a compound of Formula I-IV or a pharmaceutically acceptable salt or hydrate thereof, with one or more pharmaceutically acceptable carriers, diluents, or excipients. In some embodiments, the composition further comprises one or more therapeutic agents.

In some embodiments, the pharmaceutical composition is a tablet composition. Suitable inactive ingredients in the tablet composition include microcrystalline cellulose, croscamellose sodium, sodium stearyl fumarate, among others.

The tablet composition can include from about 25 mg to about 1400 mg, from about 50 mg to about 600 mg, or from about 50 mg to about 200 mg of the SSTR4 agonist, or a hydrate or pharmaceutically acceptable salt thereof.

In some embodiments, the pharmaceutical composition is a capsule composition. In some embodiments, the capsule composition can include the SSTR4 agonist compound, or a hydrate or pharmaceutically acceptable salt thereof, without any inactive ingredients. In some embodiments, the capsule composition can include one or more pharmaceutically acceptable carriers, diluents, or excipients. In some embodiments, the capsule composition can include microcrystalline cellulose, silica dioxide, colloidal silica dioxide, among other suitable inactive ingredients.

The capsule composition can include from about 25 mg to about 1400 mg, from about 50 mg to about 600 mg, or from about 50 mg to about 200 mg of the SSTR4 agonist, or a hydrate or pharmaceutically acceptable salt thereof. The capsule composition can include a suitable capsule, such as a gelatin capsule. The capsule can be a size 000, 00, 0, 1, or 2 depending on the total amount of material added.

In some embodiments, the pharmaceutical composition is an aqueous composition.

### Methods of Treatment

Also disclosed herein is a method of treating pain in a patient comprising administering to the patient in need of such treatment an effective amount of a compound of Formula I-IV or a pharmaceutically acceptable salt or hydrate thereof, with one or more pharmaceutically acceptable carriers, diluents, or excipients.

Also disclosed herein is a method of treating pain in a patient comprising administering to the patient in need of such treatment from about 25 mg to about 1400 mg of a compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, with one or more pharmaceutically acceptable carriers, diluents, or excipients. In some embodiments, the method of treating pain in a patient can comprise administering to the patient in need of such treatment a first amount of SSTR4 agonist of about 50 mg, about 100 mg, about 200 mg, about 300 mg, about 400 mg, or about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate. In some embodiments, additional amounts of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, can be administered in a single day. In some embodiments, the first amount can be repeated or a different amount can be administered. For example, the first amount can be 400 mg and the second amount can be 400 mg or 600 mg.

In some embodiments, the method of treating pain can comprise administering to the patient in need of such treatment from about 50 mg to about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, twice daily.

In some embodiments, the method of treating pain can comprise administering to the patient in need of such treatment from about 50 mg, about 100 mg, about 200 mg, about 300 mg, about 400 mg, about 500 mg, or about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, twice daily.

Also disclosed herein is a method of treating chronic back pain, including chronic lower back pain, in a patient comprising administering to a patient in need of such treatment an effective amount of a compound of Formula I-IV or a pharmaceutically acceptable salt or hydrate thereof, with one or more pharmaceutically acceptable carriers, diluents, or excipients.

Also disclosed herein is a method of treating chronic back pain, including chronic lower back pain, in a patient comprising administering to the patient in need of such treatment from about 25 mg to about 1400 mg of a compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, with one or more pharmaceutically acceptable carriers, diluents, or excipients. In some embodiments, the method of treating chronic back pain, including chronic lower back pain, in a patient can comprise administering to the patient in need of such treatment a first amount of SSTR4 agonist of about 50 mg, about 100 mg, about 200 mg, about 300 mg, about 400 mg, about 500 mg or about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate. In some embodiments, additional amounts of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, can be administered in a single day. In some embodiments, the first amount can be repeated or a different amount can be administered. For example, the first amount can be 400 mg and the second amount can be 400 mg or 600 mg.

In some embodiments, the method of chronic back pain, including chronic lower back pain, can comprise administering to the patient in need of such treatment from about 50 mg to about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, twice daily.

In some embodiments, the method of treating chronic back pain, including chronic lower back pain, can comprise administering to the patient in need of such treatment from about 50 mg, about 100 mg, about 200 mg, about 300 mg, about 400 mg, about 500 mg, or about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, twice daily.

Also disclosed herein is a method of treating neuropathic pain in a patient comprising administering to a patient in need of such treatment an effective amount of a compound of Formula I-IV or a pharmaceutically acceptable salt or hydrate thereof, with one or more pharmaceutically acceptable carriers, diluents, or excipients. In some embodiments, the neuropathic pain is diabetic neuropathy, diabetic peripheral neuropathic pain, polyneuropathy, distal sensory polyneuropathy, peripheral neuropathy, central neuropathy, and/or mixed neuropathy.

Also disclosed herein is a method of treating neuropathic pain, including diabetic neuropathy, diabetic peripheral neuropathic pain, polyneuropathy, distal sensory polyneuropathy, peripheral neuropathy, central neuropathy, and/or mixed neuropathy, in a patient comprising administering to the patient in need of such treatment from about 25 mg to about 1400 mg of a compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, with one or more pharmaceutically acceptable carriers, diluents, or excipients. In some embodiments, the method of treating neuropathic pain, including diabetic neuropathy, polyneuropathy, distal sensory polyneuropathy, peripheral neuropathy, diabetic peripheral neuropathic pain, central neuropathy, and/or mixed neuropathy, in a patient can comprise administering to the patient in need of such treatment a first amount of SSTR4 agonist of about 50 mg, about 100 mg, about 200 mg, about 300 mg, about 400 mg, about 500 mg, or about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate. In some embodiments, additional amounts of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, can be administered in a single day. In some embodiments, the first amount can be repeated or a different amount can be administered. For example, the first amount can be 400 mg and the second amount can be 400 mg or 600 mg.

In some embodiments, the method of treating neuropathic pain, including diabetic neuropathy, diabetic peripheral neuropathic pain, polyneuropathy, distal sensory polyneuropathy, peripheral neuropathy, central neuropathy, and/or mixed neuropathy, can comprise administering to the patient in need of such treatment from about 50 mg to about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, twice daily.

In some embodiments, the method of treating neuropathic pain, including diabetic neuropathy, diabetic peripheral neuropathic pain, polyneuropathy, distal sensory polyneuropathy, peripheral neuropathy, central neuropathy, and/or mixed neuropathy, can comprise administering to the patient in need of such treatment from about 50 mg, about 100 mg, about 200 mg, about 300 mg, about 400 mg, about 500 mg, or about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, twice daily.

Also disclosed herein is a method of treating pain associated with osteoarthritis in a patient comprising administering to a patient in need of such treatment an effective amount of a compound of Formula I-IV or a pharmaceutically acceptable salt or hydrate thereof, with one or more pharmaceutically acceptable carriers, diluents, or excipients.

Also disclosed herein is a method of treating osteoarthritic pain in a patient comprising administering to the patient in need of such treatment from about 25 mg to about 1400 mg of a compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, with one or more pharmaceutically acceptable carriers, diluents, or excipients. In some embodiments, the method of treating osteoarthritic pain in a patient can comprise administering to the patient in need of such treatment a first amount of SSTR4 agonist of about 50 mg, about 100 mg, about 200 mg, about 300 mg, about 400 mg, or about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate. In some embodiments, additional amounts of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, can be administered in a single day. In some embodiments, the first amount can be repeated or a different amount can be administered. For example, the first amount can be 400 mg and the second amount can be 400 mg or 600 mg.

In some embodiments, the method of treating osteoarthritic pain can comprise administering to the patient in need of such treatment from about 50 mg to about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, twice daily.

In some embodiments, the method of treating osteoarthritic pain can comprise administering to the patient in need of such treatment from about 50 mg, about 100 mg, about 200 mg, about 300 mg, about 400 mg, about 500 mg, or about 600 mg of the compound of Formula III, i.e. (1R,5S,6r)-N-(2-methyl-1-((3-methylpyridin-2-yl)oxy)propan-2-yl)-3-azabicyclo[3.1.0]hexane-6-carboxamide L-tartrate or Formula IV, .i.e. (1R,5S,6r)-N-(2-methyl-1-((3-methylpyridin-2-yl)oxy)propan-2-yl)-3-azabicyclo[3.1.0]hexane-6-carboxamide L-tartrate sesquihydrate, twice daily.

Also disclosed herein is a compound of Formula I-IV or a pharmaceutically acceptable salt or hydrate thereof, for use in therapy. Additionally, disclosed herein is a compound of Formula I-IV or a pharmaceutically acceptable salt or hydrate thereof, for use in the treatment of pain. Also disclosed herein is a compound of Formula I-IV or a pharmaceutically acceptable salt or hydrate thereof, for use in the treatment of chronic back pain, including chronic lower back pain. Also disclosed herein is a compound of Formula I-IV or a pharmaceutically acceptable salt or hydrate thereof, for use in the treatment of neuropathic pain. In some embodiments the neuropathic pain is diabetic neuropathy, diabetic peripheral neuropathic pain, polyneuropathy, distal sensory polyneuropathy, peripheral neuropathy, central neuropathy, and/or mixed neuropathy. Also disclosed herein is a compound of Formula I-IV or a pharmaceutically acceptable salt or hydrate thereof, for use in the treatment of pain associated with osteoarthritis.

Also disclosed herein is the use of from about 25 mg to about 1400 mg of a compound of Formula I-IV or a pharmaceutically acceptable salt or hydrate thereof, in the treatment of pain. Also disclosed herein is the use of from about 25 mg to about 1400 mg of a compound of Formula I-IV or a pharmaceutically acceptable salt or hydrate thereof, in the treatment of chronic back pain, including chronic lower back pain. Also disclosed herein is the use of from about 25 mg to about 1400 mg of a compound of Formula I-IV or a pharmaceutically acceptable salt or hydrate thereof, in the treatment of neuropathic pain. In some embodiments the neuropathic pain is diabetic neuropathy, diabetic peripheral neuropathic pain, polyneuropathy, distal sensory polyneuropathy, peripheral neuropathy, central neuropathy, and/or mixed neuropathy. Also disclosed herein is the use of from about 25 mg to about 1400 mg of a compound of Formula I-IV or a pharmaceutically acceptable salt or hydrate thereof, in the treatment of pain associated with osteoarthritis.

Also disclosed herein is the use of from about 50 mg to about 600 mg of a compound of Formula I-IV or a pharmaceutically acceptable salt or hydrate thereof, in the treatment of pain. Also disclosed herein is the use of from about 50 mg to about 600 mg of a compound of Formula I-IV or a pharmaceutically acceptable salt or hydrate thereof, in the treatment of chronic back pain, including chronic lower back pain. Also disclosed herein is the use of from about 50 mg to about 600 mg of a compound of Formula I-IV or a pharmaceutically acceptable salt or hydrate thereof, in the treatment of neuropathic pain. In some embodiments the neuropathic pain is diabetic neuropathy, diabetic peripheral neuropathic pain, polyneuropathy, distal sensory polyneuropathy, peripheral neuropathy, central neuropathy, and/or mixed neuropathy. Also disclosed herein is the use of from about 50 mg to about 600 mg of a compound of Formula I-IV or a pharmaceutically acceptable salt or hydrate thereof, in the treatment of pain associated with osteoarthritis.

Also disclosed herein is the use of about 600 mg of a compound of Formula I-IV or a pharmaceutically acceptable salt or hydrate thereof, in the treatment of pain. Also disclosed herein is the use of about 600 mg of a compound of Formula I-IV or a pharmaceutically acceptable salt or hydrate thereof, in the treatment of chronic back pain, including chronic lower back pain. Also disclosed herein is the use of about 600 mg of a compound of Formula I-IV or a pharmaceutically acceptable salt or hydrate thereof, in the treatment of neuropathic pain. In some embodiments the neuropathic pain is diabetic neuropathy, diabetic peripheral neuropathic pain, polyneuropathy, distal sensory polyneuropathy, peripheral neuropathy, central neuropathy, and/or mixed neuropathy. Also disclosed herein is the use of about 600 mg of a compound of Formula I-IV or a pharmaceutically acceptable salt or hydrate thereof, in the treatment of pain associated with osteoarthritis.

Also disclosed herein is the use of about 400 mg of a compound of Formula I-IV or a pharmaceutically acceptable salt or hydrate thereof, in the treatment of pain. Also disclosed herein is the use of about 400 mg of a compound of Formula I-IV or a pharmaceutically acceptable salt or hydrate thereof, in the treatment of chronic back pain, including chronic lower back pain. Also disclosed herein is the use of about 400 mg of a compound of Formula I-IV or a pharmaceutically acceptable salt or hydrate thereof, in the treatment of neuropathic pain. In some embodiments the neuropathic pain is diabetic neuropathy, diabetic peripheral neuropathic pain, polyneuropathy, distal sensory polyneuropathy, peripheral neuropathy, central neuropathy, and/or mixed neuropathy. Also disclosed herein is the use of about 400 mg of a compound of Formula I-IV or a pharmaceutically acceptable salt or hydrate thereof, in the treatment of pain associated with osteoarthritis.

Also disclosed herein is the use of about 200 mg of a compound of Formula I-IV or a pharmaceutically acceptable salt or hydrate thereof, in the treatment of pain. Also disclosed herein is the use of about 200 mg of a compound of Formula I-IV or a pharmaceutically acceptable salt or hydrate thereof, in the treatment of chronic back pain, including chronic lower back pain. Also disclosed herein is the use of about 200 mg of a compound of Formula I-IV or a pharmaceutically acceptable salt or hydrate thereof, in the treatment of neuropathic pain. In some embodiments the neuropathic pain is diabetic neuropathy, diabetic peripheral neuropathic pain, polyneuropathy, distal sensory polyneuropathy, peripheral neuropathy, central neuropathy, and/or mixed neuropathy. Also disclosed herein is the use of about 200 mg of a compound of Formula I-IV or a pharmaceutically acceptable salt or hydrate thereof, in the treatment of pain associated with osteoarthritis.

In addition, also disclosed herein is the use of a compound of Formula I-IV or a pharmaceutically acceptable salt or hydrate thereof, for the manufacture of a medicament for the treatment of a disease or condition selected from pain, chronic back pain, including chronic lower back pain, neuropathic pain and pain associated with osteoarthritis. In some embodiments the neuropathic pain is diabetic neuropathy, diabetic peripheral neuropathic pain, polyneuropathy, distal sensory polyneuropathy, peripheral neuropathy, central neuropathy, and/or mixed neuropathy.

In some embodiments, the first or effective amount of the SSTR4 agonist can be administered at least once a day, from once to four times a day, at least twice a day, once daily, twice daily, every 24 hours, every 12 hours, every 8 hours, every 6 hours, or every 4 hours.

Patients that are in need of a treatment for diabetic neuropathy can include any patient that meets one or more of the following characteristics: have a visual analog scale (VAS) pain value 40 or more and less than 95 prior to starting therapy, have a history of daily pain for at least 12 weeks based on participant report or medical history, have a value of 30 or less on the Pain Catastrophizing Scale, have a body mass index of less than 40 kg/m2 (inclusive), have a daily symmetrical foot pain secondary to peripheral neuropathy present for at least 6 months and as diagnosed through use of a validated tool, for example the Michigan Neuropathy Screening Instrument Part B 3 or more, have a history and current diagnosis of type 1 or type 2 diabetes mellitus, and/or have a stable glycemic control as indicated by a glycated hemoglobin level of 11 or less prior to starting therapy.

### Definitions

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “hydrate” refers to a solid adduct containing a compound, or a salt thereof, and water, wherein water molecules are incorporated into the crystal lattice of said compound, or salt thereof. As used herein, the term “sesquihydrate” refers to a hydrate of a compound, or salt thereof, wherein the stoichiometric ratio of water to compound, or salt thereof, is 1.5:1.

As used herein, the terms “treating” or “to treat” includes restraining, slowing, stopping, or reversing the progression or severity of an existing symptom or disorder.

As used herein, the term “patient” refers to a mammal, such as a mouse, guinea pig, rat, dog, or human. It is understood that the preferred patient is a human.

As used herein, the term “effective amount” refers to the amount or dose of compound of the invention which, upon single or multiple dose administration to the patient, provides the desired effect in the patient under diagnosis or treatment.

An effective amount can be readily determined by one skilled in the art by the use of known techniques. In determining the effective amount for a patient, a number of factors are considered, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The terms “treatment” and “treating” are intended to refer to all methods wherein there may be a slowing, interrupting, arresting, controlling, or stopping of the progression of an existing disorder and/or symptoms thereof, but does not necessarily indicate a total elimination of all symptoms.

A “pharmaceutically acceptable carrier, diluent, or excipient” is a medium generally accepted in the art for the delivery of biologically active agents to mammals, e.g., humans.

A “dose” refers to a predetermined quantity or unit dose of Formula I, (1R,5S,6r)-N-(2-methyl-1-((3-methylpyridin-2-yl)oxy)propan-2-yl)-3-azabicyclo[3.1.0]hexane-6-carboxamide, calculated to produce the desired therapeutic effect in a patient. As used herein “mg” refers to milligram. As used herein, dose ranges and doses provided of represent the weight of the active pharmaceutical ingredient, Formula I, (1R,5S,6r)-N-(2-methyl-1-((3-methylpyridin-2-yl)oxy)propan-2-yl)-3-azabicyclo[3.1.0]hexane-6-carboxamide, regardless of the form in which it is provided, such as the free base, salt, cocrystalline form, or any other composition or form.

The term “about” as used herein, means in reasonable vicinity of the stated numerical value, such as plus or minus 10% of the stated numerical value.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

As used herein, the term “C₁-Cₙ thioether” refers to a straight, or branched chain saturated hydrocarbon containing 1 to n carbon atoms containing a terminal “S” in the chain, i.e., —S(alkyl), wherein the thioether group can be attached to the desired location at the sulfur atom. The term “C₁-Cₙ thioether” also refers to a cycloalkyl or aryl radical containing a terminal “S” in the molecule, i.e. —S(aryl), wherein the thioether group can be attached to the desired location at the sulfur atom. The term “C₁-Cₙ thioether” can include a saturated hydrocarbon chain and either a cycloalkyl or aryl, i.e. —S—CH₂-(aryl), with n referring to the number of total carbon atoms in the substituent.

As used herein, the term “C₁-Cₙ ether” refers to a straight, or branched chain saturated hydrocarbon containing 1 to n carbon atoms containing a terminal “O” in the chain, i.e., —O(alkyl), wherein the ether group can be attached to the desired location at the oxygen atom. The term C₁-Cₙ ether also refers to a cycloalkyl or aryl radical containing a terminal “O” in the molecule, i.e. —O(aryl), wherein the ether group can be attached to the desired location at the oxygen atom. The term “C₁-Cₙ ether” can also include a saturated hydrocarbon chain and either a cycloalkyl or aryl, i.e. —O—CH₂-(aryl), with n referring to the number of total carbon atoms in the substituent.

As used herein, the term “cycloalkyl” means a radical derived from a non-aromatic monocyclic or polycyclic ring comprising carbon and hydrogen atoms. The cycloalkyl can have one or more carbon-carbon double bonds in the ring as long as the ring is not rendered aromatic by their presence. The cycloalkyl group can be unsubstituted or substituted with from one to three suitable substituents, which are well known to a person of ordinary skill in the art. The cycloalkyl group can be referred to by the number of total carbon atoms in the monocyclic or polycyclic ring. For example, a C₃ to C₇ cycloalkyl includes cycloalkyl radical group with 3, 4, 5, 6, or 7 carbon atoms.

As used herein, the term “heterocycloalkyl” means a radical derived from a non-aromatic monocyclic or polycyclic ring comprising one or more carbon atoms and one or more heteroatoms, such as nitrogen, oxygen, and sulfur. A heterocycloalkyl group can have one or more carbon-carbon double bonds or carbon-heteroatoms double bonds in the ring as long as the ring is not rendered aromatic by their presence. Examples of heterocycloalkyl groups include aziridinyl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl, and pyranyl. A heterocycloalkyl group can be unsubstituted or substituted with one or two suitable substituents. The heterocycloakyl group can be referred to by the number of total atoms in the monocyclic or polycyclic ring. For example, a four member to seven member heterocycloalkyl includes four, five, six, or seven members (including carbon atoms and heteroatoms).

As used, herein, the term “aryl” means a radical derived from an aromatic monocyclic or polycyclic ring including only carbon atoms in the monocyclic or polycyclic ring. The aryl group can be unsubstituted or the aryl group can be substituted with from 1 to 5 suitable substituents, which are well known to a person of ordinary skill in the art. The aryl group can be referred to by the number of total carbon atoms in the monocyclic or polycyclic ring. For example, a C₅ to C₇ aryl includes an aryl radical group with 5, 6, or 7 carbon atoms.

As used herein, the term “heteroaryl” means a radical derived from an aromatic monocyclic or polycyclic ring including one or more carbon atoms and one or more heteroatoms in the monocyclic or polycyclic ring. The heteroaryl group can be unsubstituted or the heteroaryl group can be substituted with from 1 to 5 suitable substituents, which are well known to person of ordinary skill in the art. The heteroaryl group can be referred to by the number of total atoms in the monocyclic or polycyclic ring. For example, a four member to seven member heteroaryl includes four, five, six, or seven members (including carbon atoms and heteroatoms).

### EXAMPLES

Certain abbreviations are defined as follows: “DCM” refers to dichloromethane; “DMAP” refers to 4-dimethylaminopyridine; “DMF” refers to dimethylformamide; “EtOAC” refers to ethyl acetate; “EtOH” refers to ethanol; “hr/hrs” refers to hour/hours; “MeCN” refers to acetonitrile; “MeOH” refers to methanol; “MTBE” refers to methyl tert-butyl ether; “MeTHF” refers to methyltetrahydrofuran; “OTf” refers to trifluoromethanesulfonate; “Ph₂PK” refers to potassium diphenylphosphide; “TEA” refers to triethylamine; “TFA” refers to trifluoroacetic acid; and “THF” refers to tetrahydrofuran.

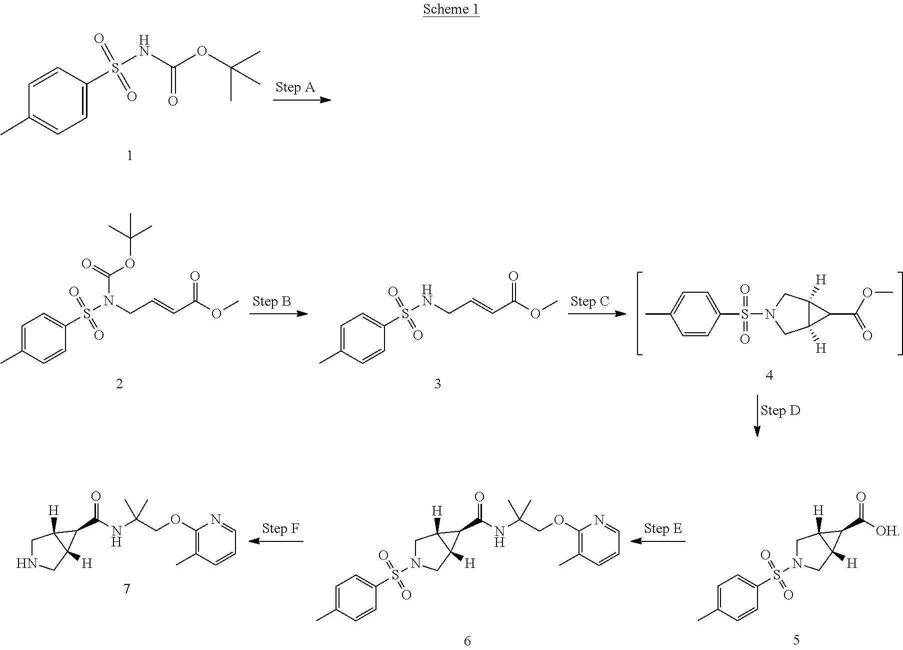

### Commercial Synthesis of an SSTR4 Agonist

Scheme 1, step A depicts a coupling mixing compound (1) with methyl 4-bromobut-2-enoate using an appropriate base such as potassium carbonate and potassium iodide in a suitable solvent such as MeCN at a suitable temperature (e.g., 30° C.) to give compound (2). Step B shows the deprotection of compound (2) using an acid such as TFA in solvents such as MeCN to give compound (3). Step C shows a cyclization mixing compound (3) using (2-haloethyl)diphenylsulfonium such as (2-bromoethyl)diphenylsulfonium triflate in presence of base KOH, KF, and a suitable solvent such as 2-MeTHF to give compound (4). In step D, water and a base such as LiOH are used to convert compound (4) to compound (5). Step E shows a coupling mixing compound (5) with 2-methyl-1-((3-methylpyridin-2-yl)oxy)propan-2-amine in presence of a catalyst such as COCl₂ and solvents such as DMF, toluene, and DCM to give compound (6). Step F shows conversion of compound (6) to compound (7) by reacting compound (6) with Ph₂PK in a solvent such as MTBE under a suitable temperature (e.g., heating from 60 to 70° C.) to give compound (7).

### Preparation 1

### Methyl (E)-4-((4-methylphenyl)sulfonamido)but-2-enoate

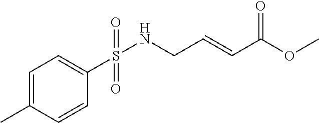

Methyl 4-bromobut-2-enoate (36.29 g, 202.7 mmol) was dissolved in MeCN (500 mL) at 15-25° C. tert-Butyl tosylcarbamate (50.00 g, 184.3 mmol) was added at 15-25° C. K₂CO₃ (30.57 g, 221.2 mmol) and KI (3.06 g, 202.7 mmol) were added to the solution at 15-25° C., and warmed under nitrogen at 30° C. for 20 hrs. The solution was cooled to 20° C. and the mixture filtered. The filtered residue was washed with MeCN (100 mL) to give methyl (E)-4-((N-(tert-butoxycarbonyl)-4-methylphenyl)sulfonamido)but-2-enoate. TFA (101.03 g, 886.06 mmol) was added to the methyl (E)-4-((N-(tert-butoxycarbonyl)-4-methylphenyl)sulfonamido)but-2-enoate in MeCN solution (483.72 g, 143 mmol) and heated to 55-60° C. for 16 hrs. The reaction solution was concentrated in vacuo to ˜50 mL and solvent exchanged with toluene (2×250 mL). Toluene (500 mL) was added followed by EtOAc (50 mL) at 15-25° C., and heated to 60° C. for 1 hr., then cooled to 0° C. for 12 hrs. The solution was filtered and the wet cake was rinsed with n-heptane (50 mL). The cake was dried in vacuum at 50° C. to give the title compound (37.85 g, 74.4%) as a white solid. ¹H NMR (CDCl₃) δ 7.68 (d, J=8.0 Hz, 2H) 7.25 (d, J=8.0 Hz, 2H) 6.71 (dt, J=15.6, 5.2 Hz, 1H) 5.88 (dt, J=15.6, 1.6 Hz, 1H) 4.55 (t, J=6.4 Hz, 1H) 3.71-3.67 (m, 2H) 3.65 (s, 3H) 2.37 (s, 3H); HRMS (ESI⁺) Calculated for [C₁₂H₁₅NO₄S+H]⁺: 270.0795, Found: 270.0788 (M+H).

### Preparation 2

### (1R,5S,6r)-3-Tosyl-3-azabicyclo[3.1.0]hexane-6-carboxylic acid

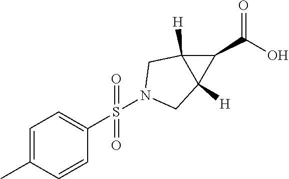 [SMILES: Cc1ccc(S(=O)(=O)N2C[C@@H]3[C@H](C2)[C@H]3C(=O)O)cc1]

Methyl (E)-4-((4-methylphenyl)sulfonamido)but-2-enoate (51.90 g) was dissolved in 2-MeTHF (600 mL) at 0° C. Added (2-bromoethyl)diphenylsulfonium triflate (36.50 g), KF (6.47 g), KOH (18.75 g) to the solution at 0° C. Warmed the solution to 15° C. for 22 hrs. then 30° C. for 3 hrs. Added water (100 mL) and MeOH (100 mL) into the solution. Added LiOH·H₂O (4.77 g) and stirred at 30° C. for 16 hrs. Cooled to 15-25° C. and added n-heptane (100 mL). Stirred at 15-25° C. for 10 min. Separated and collected the aqueous phase and washed the aqueous phase with n-heptane/2-MeTHF (50 mL/200 mL×2). Concentrated the aqueous phase in vacuo to ˜50 mL and added 3M aq. HCl dropwise to adjust pH to 1˜2. Stirred the mixture at 20-30° C. for 2 hrs. Filtered the solution and rinsed through with EtOH/H₂O (15 mL 1:4). Dried the wet cake at 45° C. for 8-10 hrs. to give the title compound as white solid (20.37 g, 65%)¹H NMR (CDCl₃) δ 7.67 (d, J=8.2 Hz, 2H) 7.34 (d, J=8.2 Hz, 2H) 3.63 (d, J=9.4 Hz, 2H) 3.12 (d, J=9.4 Hz, 2H) 2.46-2.40 (m, 4H) 2.07-2.01 (m, 2H); HRMS (ESI⁺) Calcd. for [C₁₃H₁₅NO₄S+H]⁺: 282.0795, Found: 282.0795 (M+H).

### Preparation 3

### 2-Methyl-1-((3-methylpyridin-2-yl)oxy)propan-2-amine

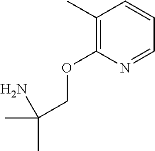

2-Amino-2-methylpropan-1-ol (50.14 g, 562.5 mmol) was dissolved in toluene (250 mL) at 15-25° C. Added potassium tert-butoxide (60.59 g, 534.6 mmol) to the solution and warmed to 30-40° C. Added 2-fluoro-3-methylpyridine (50.00 g, 450.0 mmol) to the solution and warmed to 80° C. Stirred the mixture for 18 hrs. at 80° C. Cooled the mixture to 15-25° C., added water (100 mL) and stirred for 30 min. Separated the aqueous layer and washed the organics with 10% aq. NaCl (300 mL×3). Added toluene (250 mL) to the organics and concentrated in vacuo to give the title compound as a liquid (107.50 g, 98%). ¹H NMR (CDCl₃) δ 7.92-7.82 (m, 1H) 7.32-7.22 (m, 1H) 6.74-6.66 (m, 1H) 3.97 (s, 3H) 2.14 (s, 3H) 1.15 (s, 6H).

### Preparation 4

### (1R,5S,6r)-N-(2-Methyl-1-((3-methylpyridin-2-yl)oxy)propan-2-yl)-3-tosyl-3-azabicyclo[3.1.0]hexane-6-carboxamide

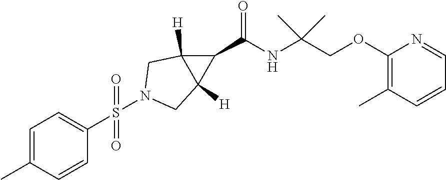 [SMILES: Cc1ccc(S(=O)(=O)N2C[C@@H]3[C@H](C2)[C@H]3C(=O)NC(C)(C)COc2ncccc2C)cc1]

Dissolved (1R,5S,6r)-3-tosyl-3-azabicyclo[3.1.0]hexane-6-carboxylic acid (32.33 g, 88.2% purity) in toluene (320 mL) at 15˜25° C. Added DMF (741.0 mg) and (COCl)₂ (19.20 g) to the solution and heated to 45-55° C. for 3-4 hrs. Concentrated the mixture in vacuo and exchanged with THF (100 mL×2). Added THE (320 mL) and cooled to 0-10° C. Added 2-methyl-1-((3-methylpyridin-2-yl)oxy)propan-2-amine (23.60 g), TEA (30.80 g), DMAP (620.0 mg) at 0-10° C. then warmed to 15-25° C. for 2˜4 hrs. Concentrated the mixture in vacuo and solvent exchanged with EtOH (140 mL×2). Concentrated in vacuo to 140 mL and heated to 50° C. until the solid was dissolved. Added water (210 mL) dropwise into the solution at 40° C. Cooled the solution to 10° C. for 14 hrs. Filtered and rinsed with EtOH/H²O (75 mL, 1:1.5). Dried the wet cake at 45° C. for 20 hrs. to give the title compound as a solid (41.87 g, 87.4%). ¹H NMR (CDCl₃) δ 7.94-7.87 (m, 1H) 7.60 (d, J=8.0 Hz, 2H) 7.37 (d, J=6.6 Hz, 1H) 7.26 (d, J=8.0 Hz, 2H) 6.78 (dd, J=6.6, 5.0 Hz, 1H) 6.48 (s, 1H) 4.25 (s, 2H) 3.52 (d, J=9.4 Hz, 2H) 2.95 (d, J=9.4 Hz, 2H) 2.39-2.33 (m, 3H) 2.17 (s, 3H) 1.84 (s, 2H) 1.39 (s, 6H); HRMS (ESI⁺) Calcd. for [C₂₃H₂₉N₃O₄S+H]⁺: 444.1952, Found: 444.2089 (M+H).

### Example 1

### (1R,5S,6r)-N-(2-Methyl-1-((3-methylpyridin-2-yl)oxy)propan-2-yl)-3-azabicyclo[3.1.0]hexane-6-carboxamide

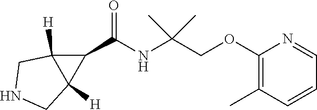 [SMILES: Cc1cccnc1OCC(C)(C)NC(=O)[C@H]1[C@@H]2CNC[C@@H]21]

Dissolved (1R,5S,6r)-N-(2-methyl-1-((3-methylpyridin-2-yl)oxy)propan-2-yl)-3-tosyl-3-azabicyclo[3.1.0]hexane-6-carboxamide (5.00 g) in MTBE (50 mL) under nitrogen. Cooled to −70-60° C., and added dropwise 1M Ph₂PK in THE (56 mL, 56 mmol) into the solution. Stirred the mixture for 6-8 hrs. at −70-60° C. Added 2M aq. HCl (50 mL) to the solution allowing the temperature to rise to 15-25° C. Separated and collected the aqueous phase and washed with 2-MeTHF (50 mL×3). Added 2-MeTHF (50 mL) and adjusted the pH to 8˜9 with K₂CO₃ powder. Separated and extracted the aqueous phase with 2-MeTHF (50 mL). Combined the organics and concentrated in vacuo to give the title compound as a yellow-brown solid (3.05 g, 89.4%)¹H NMR (CDCl₃) δ 8.01-7.92 (m, 1H) 7.45-7.36 (m, 1H) 6.81 (dd, J=7.0, 5.0 Hz, 1H) 6.39 (s, 1H) 4.31 (s, 2H) 3.09-2.89 (m, 4H) 2.21 (s, 3H) 1.94-1.86 (m, 2H) 1.69 (s, 1H) 1.47 (s, 6H) 1.12 (t, J=2.8 Hz, 1H); HRMS (ESI⁺) Calcd. for [C₁₆H₂₃N₃O₂+H]⁺: 290.1863, Found: 290.1917 (M+H).

### Example 2

### (1R,5S,6r)-N-(2-Methyl-1-((3-methylpyridin-2-yl)oxy)propan-2-yl)-3-azabicyclo[3.1.0]hexane-6-carboxamide L-tartrate

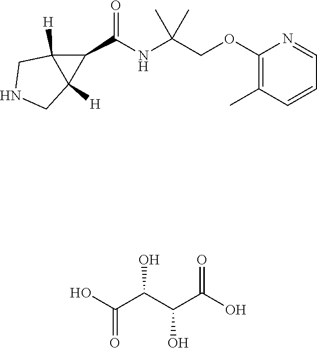 [SMILES: Cc1cccnc1OCC(C)(C)NC(=O)[C@H]1[C@@H]2CNC[C@@H]21 ; O=C(O)[C@H](O)[C@@H](O)C(=O)O]

To (1R,5S,6r)-N-(2-methyl-1-((3-methylpyridin-2-yl)oxy)propan-2-yl)-3-azabicyclo[3.1.0]hexane-6-carboxamide (5.5 g, 18.4 mmol) was added IPA (68 mL) and water (2 ml). The mixture was heated to 65° C., at which point dissolution occurs. L-Tartaric acid (2.86 g, 19.1 mmol) in IPA (34 mL) and water (1.5 mL) were then added to the solution. The solution was then allowed to cool to ambient temperature overnight. The resulting white solid was isolated by vacuum filtration and rinsed with ice-cold IPA (20 mL) to give the title compound (5.7 g, 70%).

### Example 3

### (1R,5S,6r)-N-(2-Methyl-1-((3-methylpyridin-2-yl)oxy)propan-2-yl)-3-azabicyclo[3.1.0]hexane-6-carboxamide citrate

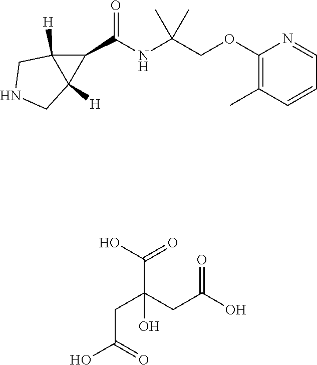 [SMILES: Cc1cccnc1OCC(C)(C)NC(=O)[C@H]1[C@@H]2CNC[C@@H]21 ; O=C(O)CC(O)(CC(=O)O)C(=O)O]

(1R,5S,6r)-N-(2-Methyl-1-((3-methylpyridin-2-yl)oxy)propan-2-yl)-3-azabicyclo[3.1.0]hexane-6-carboxamide (10.8 g, 33 mmol) was dissolved in absolute EtOH (200 mL) while stirring at 300 rpm at 60° C. This solution was filtered through a 0.65 μm nylon filter to yield a clear solution. The solution was stirred for 5 min upon which solid precipitation occurs. A solution of citric acid (7.06 g, 36 mmol) dissolved in absolute EtOH (60 mL) at 60° C. was prepared. The citric acid solution was added slowly at 60° C. The mixture was filtered through a 0.45 μm syringe filter maintained at 60° C. Heating was then terminated and the mixture stirred at 500 rpm while gradually cooling to ambient temperature. Upon complete equilibration to ambient temperature, a very thick white slurry (cake) was obtained. The flask was rinsed with absolute EtOH (5×10 mL) to rinse the cake. The cake solid was isolated on a nylon membrane under vacuum, dried under nitrogen, then overnight at 70° C. under vacuum to give the title compound as a white solid (16.8 g, 98%).

### Example 4

### (1R,5S,6r)-N-(2-Methyl-1-((3-methylpyridin-2-yl)oxy)propan-2-yl)-3-azabicyclo[3.1.0]hexane-6-carboxamide L-malate

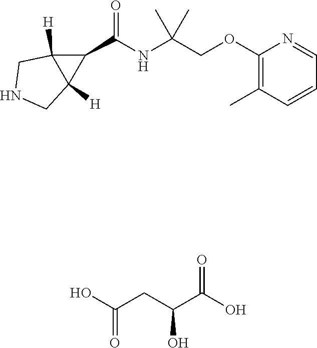 [SMILES: Cc1cccnc1OCC(C)(C)NC(=O)[C@H]1[C@@H]2CNC[C@@H]21 ; O=C(O)C[C@H](O)C(=O)O]

(1R,5S,6r)-N-(2-Methyl-1-((3-methylpyridin-2-yl)oxy)propan-2-yl)-3-azabicyclo[3.1.0]hexane-6-carboxamide (25 g, 88 mmol) was added to IPA (100 mL) while stirring at ˜400 rpm. The sample was heated to 60° C. L-Malic acid solution in water (14.6 mL, 109 mmol) was then added. A clear yellowish solution was formed and the solution was cooled to ambient temperature. Oiling was observed, so the phase separation was evaporated to dryness under nitrogen stream. The solid residue was suspended in acetone and water for a recrystallization at 55° C. The 25 g freebase equivalent material was recrystallized in acetone (200 mL) and water (15 mL). The solid was isolated from the reactor vessel at ambient temperature via vacuum filtration through a Buchner funnel. The resulting white cake was rinsed with acetone and dried at 50° C. under vacuum to give the title compound (21 g, 57%).

### Example 5

### (1R,5S,6r)-N-(2-Methyl-1-((3-methylpyridin-2-yl)oxy)propan-2-yl)-3-azabicyclo[3.1.0]hexane-6-carboxamide L-tartrate sesquihydrate

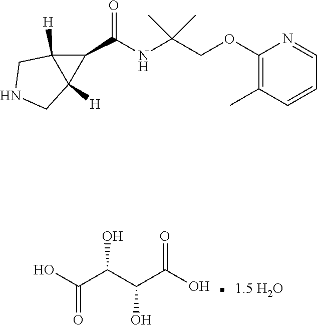 [SMILES: Cc1cccnc1OCC(C)(C)NC(=O)[C@H]1[C@@H]2CNC[C@@H]21 ; O=C(O)[C@H](O)[C@@H](O)C(=O)O]

(1R,5S,6r)-N-(2-Methyl-1-((3-methylpyridin-2-yl)oxy)propan-2-yl)-3-azabicyclo[3.1.0]hexane-6-carboxamide L-tartrate (60 g, 136.5 mmol) was transferred into a 250 mL reactor vessel and THF/water 95:5 v/v was added to a volume of 225 mL. The mixture was heated to 60° C. and water was added in 1 mL aliquots to fully dissolve the starting material (total 8 mL of water). The reactor was allowed to cool naturally, and the mixture was allowed to stir at RT over the weekend. The resulting crystals were isolated by vacuum filtration and air-dried for several days. The resulting solid was sieved to give the title compound (42.3 g, 66%). Example 5 was also synthesized in U.S. patent application Ser. No. 17/944,429, which is herein incorporated by reference in its entirety.

### Example 6

### Phase 1 Clinical Studies of (1R,5S,6r)-N-(2-Methyl-1-((3-methylpyridin-2-yl)oxy)propan-2-yl)-3-azabicyclo[3.1.0]hexane-6-carboxamide L-tartrate sesquihydrate (Example 5)

(1R,5S,6r)-N-(2-Methyl-1-((3-methylpyridin-2-yl)oxy)propan-2-yl)-3-azabicyclo[3.1.0]hexane-6-carboxamide L-tartrate sesquihydrate (Example 5) is currently in phase 2 clinical trials for the treatment of diabetic neuropathy, diabetic peripheral neuropathic pain, and mixed neuropathic pain. Prior data from 2 phase 1 studies (J2P-MC-LXBB (LXBB) and LXBA), provide evidence of the concept for the safe use of Example 5 in the treatment and/or relief of certain types of pain, such as neuropathic pain, in a patient, the treatment comprising administering to the patient a dose of about 50 mg to about 600 mg of Example 5 either once a day or twice a day.

Example 5, if approved by a corresponding regulatory agency, would be a first-in-class SSTR4 agonist. There is no available biomarker or pharmacodynamic test to predict the correct dose using only in vitro testing. Thus, it is believed that the establishment of a safe and efficacious dose for the treatment of pain with an SSTR4 agonist could only be determined with in vivo testing.

A total of 116 healthy subjects received at least one dose of Example 5 in phase 1 studies LXBB and LXBA. TABLE 1 summarizes the overview of the completed phase 1 pharmacology studies.

The single-dose pharmacokinetics (PK) of Example 5 were evaluated at a dose range of from 20 to 400 mg. Across this dose range, the median time to peak plasma concentrations (tₘₐₓ) of Example 5 ranged from 2 to 3 hours post dose and the mean terminal half-life (t_{1/2}) ranged from about 11 to 13 hours. The apparent total body clearance (CL/F) ranged from about 21 to 24 L/h. Renal clearance was approximately 10 L/h, with about 39 to 45% of the administered oral dose excreted as unchanged Example 5 in urine, and plasma concentrations of Example 5 generally increased in a dose-proportional manner and exhibited an inter-subject variability coefficient of variation of <25%.

The multiple-dose PK of Example 5 were evaluated at doses of 200 mg, 400 mg, and 600 mg, twice daily. The median time to tₘₐₓ ranged from 1 to 3 hours post dose. The accumulation ratio (R_{A}) of Example 5 ranged from about 1 to 2, which was consistent with the t_{1/2} of Example 5. Additionally, steady-state Cₘₐₓ and AUC₀₋₂₄ inter-subject coefficient of variations were <25%. This is summarized in TABLE 2.

The safety and tolerability of Example 5, were initially based on the 2 completed Phase 1 studies (LXBB and LXBA) and later confirmed with 3 completed Phase 2 studies (OA03, BP03, and NP03, as disclosed below).

TABLE 1
Overview of Completed Phase 1 Pharmacology Studies

| Study | Objectives | Study Design | Dosage and Regimen | Number of Subjects |
|---|---|---|---|---|
| LXBB | Safety and | Part 1 SAD: 7 cohorts | Part 1 | Total treated = 46 males |
| | PK | Cohorts 1 to 6: | Cohorts 1 to 6: 20 to 400 | and 43 females |
| | | Randomized, double- | mg | Part 1 |
| | | blind, placebo-controlled | Cohort 7: 100 mg | 20 mg = 6 |
| | | Cohort 7 (food effect): | Part 2 | 40 mg = 6 |
| | | Randomized, open-label, | Cohort 1: 100 mg QD | 100 mg = 6 |
| | | 2-period crossover (Fed | Cohort 2: 100 mg BID | 200 mg = 6 |
| | | vs. Fasted) | Cohort 3: 140 mg BID | 300 mg = 6 |
| | | Part 2 MAD: 4 cohorts | Cohort 4: 380 mg/day | 400 mg = 6 |
| | | Cohorts 1 to 4: young | (200 mg QAM and 180 | Placebo = 6 |
| | | healthy subjects | mg QPM) | 100 mg = 12 |
| | | Cohort 5: elderly | Cohort 5: | Part 2 |
| | | subjects (5 A, B, C | A: 40 mg QD | 100 mg QD = 8 |
| | | groups). Randomized, | B: 100 mg QD | 100 mg BID = 8 |
| | | double-blind, placebo- | C: 200 mg QD | 140 mg BID = 8 |
| | | controlled | | 380 mg/day = 8 |
| | | | | Placebo = 8 |
| | | | | 40 mg = 3 |
| | | | | 100 mg = 3 |
| | | | | 200 mg = 3 |
| | | | | Placebo = 3 |
| LXBA | Safety, | 3 cohorts ascending dose | Ex. 5/Placebo capsule | Ex. 5 |
| | tolerability, | levels; Subject- and | Cohort 1: 200 mg Q12H | 200 mg Q12H = 6 |
| | PK, and | investigator-blind, | Cohort 2: 400 mg Q12H | 400 mg Q12H = 6 |
| | PD of | randomized, placebo- | Cohort 3: 600 mg Q12H | 600 mg Q12H = 6 |
| | multiple | controlled | | Placebo = 6 |
| | daily | | | |
| | doses of | | | |
| | Ex. 5 in | | | |
| | healthy | | | |
| | subjects | | | |

Abbreviations:
BID = twice daily,
MAD = multiple-ascending dose,
PD = pharmacodynamics,
PK = pharmacokinetics,
Q12H = every 12 hours,
QAM = every morning,
QD = once daily,
QPM = every evening,
SAD = single-ascending dose

TABLE 2
Example 5 Steady-State Plasma PK Parameters After
Repeated Administration in Healthy Subjects
Geometric Mean (CV %)

| | 400 mg | | 800 mg | | 1200 mg | |
|---|---|---|---|---|---|---|
| PK Parameter | (n = 6) | | (n = 6) | | (n = 6) | |
| (Unit) | 200 mg BID | | 400 mg BID | | 600 mg BID | |
| Cₘₐₓ (ng/mL) | 1620 | (14) | 3240 | (18) | 5210 | (15) |
| Tₘₐₓ (h)^{c} | 1.01 | (1.00, 2.00) | 2.00 | (1.00, 2.00) | 1.55 | (0.50, 4.00) |
| AUC_{(0-τ)} | 11,500 | (6%) | 25,800 | (18%) | 38,700 | (14%) |
| (h × ng/mL) | | | | | | |
| AUC₍₀₋₂₄₎ | 23,000^{a} | (6%) | 51,000^{a} | (18%) | 77,400^{a} | (14%) |
| (h × ng/mL) | | | | | | |
| R_{A} | 1.49 | (13) | 1.81 | (7) | 1.64 | (9) |

Abbreviations:
AUC₀₋₂₄ = area under the plasma concentration-time curve from time 0 to 24 hours;
AUC_{(0-τ)} = area under the plasma concentration-time curve over 1 dosing interval;
BID = approximately 12 hours apart;
Cₘₐₓ = maximum observed plasma concentration;
CV % = % coefficient of variation;
PK = pharmacokinetics;
QD = once daily;
R_{A} = accumulation ratio based on AUC_{(0-τ)}, where tau = 12 hours;
tₘₐₓ = time to reach maximum observed plasma concentration.
^{a}AUC₀₋₂₄ = 2 × AUC_{(0-τ)}, where tau = 12 hours.

Phase 1 Study LXBB was a first-in-human single-ascending dose/multiple-ascending dose (SAD/MAD) study in 89 healthy subjects (TABLE 1). In the completed SAD and MAD study, Example 5 was well tolerated. All adverse events (AEs) were considered mild or moderate in severity. No deaths or serious adverse events (SAEs) were reported. One subject was discontinued due to the nondrug-related AE of pyrexia. In the SAD study, 1 subject each reported AEs of temporomandibular joint syndrome and tension headache. In the food effect study, 1 subject reported dry eye and nausea. In the Younger Group 10-day multiple-dose exposure study, the most commonly reported AEs were headache (2 subjects), diarrhea (2 subjects), nausea, rhinitis, and orthostatic hypotension (1 subject each). In the Elderly Group 10-day multiple-dose exposure study, the most commonly reported AEs were headache (5 subjects), pain in extremity (1 subject), vessel puncture site bruise (1 subject), dizziness (1 subject), and orthostatic hypotension (1 subject).

The mean PR interval increase from baseline was 13 msec in subjects receiving single 400-mg dose (SAD); the highest PR interval change from baseline in an individual subject was 45 msec (after the 300-mg dose in MAD). There were no clinically significant changes in chemistry, hematology, or coagulation parameters.

Study LXBA is a completed study and consisted of 2 parts, Part A and Part B (TABLE 1). Part A of the study had 18 healthy subjects in 3 dosing cohorts, and Part B had 9 healthy subjects exposed to Example 5. Safety data indicated that Example 5 was generally well tolerated. The majority of AEs were of mild severity. No deaths, SAEs, or discontinuations due to AEs have been reported. In Part A, all treatment-emergent adverse events (TEAEs) were mild in severity except for 1 moderate event of presyncope. Constipation and insomnia (2 events each) were the only TEAEs reported in >1 subject. In Part B, the reported TEAEs were dysgeusia, nausea, and vomiting, which were all mild in severity.

In total, the results demonstrated in the two phase 1 studies established a safety profile for Example 5 in a dose amount of from 20 mg to 1200 mg per day and/or from 20 mg to 600 mg per dose in a total of 116 healthy subjects.

### Example 7

### Capsule Compositions

TABLE A shows the capsule compositions used in the clinical studies.

| | 50 mg Dose | 50 mg Dose | 200 mg Dose |
|---|---|---|---|
| | Unit | Adjusted | Unit |
| | Formula | Unit | Formula |
| | (mg) | Formula | (mg) |
| (1R,5S,6r)-N-(2-Methyl- | 50 | 75.64 | 200 |
| 1-((3-methylpyridin-2- | | | |
| yl)oxy)propan-2-yl)-3- | | | |
| azabicyclo[3.1.0]hexane- | | | |
| 6-carboxamide L-tartrate | | | |
| sesquihydrate (Example 5) | | | |
| Microcrystalline | 249.44 | 223.8 | — |
| Cellulose | | | |
| PH-102 | | | |
| Colloidal Silica Dioxide | 4.56 | 4.56 | — |
| Aerosil 200 Pharma | | | |
| Total | 304 | 304 | 200 |
| Vacant Gelatin Capsules | 1 EA | 1 EA | 1 EA |
| Size 0, CS WHITE | | | |
| OPAQUE Cap & Body | | | |
| 0999 | | | |

### Example 8

### Phase 2 Clinical Studies of (1R,5S,6r)-N-(2-Methyl-1-((3-methylpyridin-2-yl)oxy)propan-2-yl)-3-azabicyclo[3.1.0]hexane-6-carboxamide L-tartrate sesquihydrate Example 5

Example 5 has been evaluated in 3 randomized, placebo-controlled, Phase 2 osteostudies study OA03 for the treatment of knee GA pain, study BP03 for the treatment of CLBP, and study NP03 for the treatment of DPNP. An overview of the 3 Phase 2 studies is shown in TABLE 3. TABLE 4 shows the population studied in the 3 Phase 2 studies.

TABLE 3
Randomized, Placebo-Controlled, Phase 2 Clinical Trials
to Evaluate Example 5 for Certain Indications

| Study Identifier | | Phase and Study | | Number of |
|---|---|---|---|---|
| and Indication | Objectives | Design | Dosing Regimen | Participants |
| H0P-MC-OA03 | Efficacy, PK, | An 8-week, | Example | All treated: 201 |
| Osteoarthritis Pain | Safety, and | randomized, | 5/Placebo: | Example 5: 135 |
| (OA) | Health | double-blind, | Maximum 3 | Placebo: 66 |
| | Outcomes of | placebo-controlled | capsules (200 mg | |
| | Example 5 vs. | study comparing | each) Q12H for a | |
| | Placebo | Example 5 versus | total of 600 mg | |
| | | placebo in patients | | |
| | | with OA in the knee | | |
| H0P-MC-BP03 | | An 8-week, | Example | All treated: 152 |
| Chronic Low | | randomized, | 5/Placebo: | Example 5: 102 |
| Back Pain (CLBP) | | double-blind, | Maximum 3 | Placebo: 50 |
| | | placebo-controlled | capsules (200 mg | |
| | | study comparing | each) Q12H for a | |
| | | Example 5 versus | total of 600 mg | |
| | | placebo in patients | | |
| | | with CLBP | | |
| H0P-MC-NP03 | | An 8 week, | Example | All treated: 68 |
| Diabetic | | randomized, | 5/Placebo: | Example 5: 45 |
| peripheral | | double-blind, | Maximum 3 | Placebo: 23 |
| neuropathy | | placebo-controlled | capsule (200 mg | |
| (DPNP) | | study comparing | each) Q12H for a | |
| | | Example 5 versus | total of 600 mg | |
| | | placebo in patients | | |
| | | with DPNP | | |

TABLE 4
Study Population of Phase 2 Studies

| Study Identifier and Indication | Study Population |
|---|---|
| H0P-MC-OA03 | Participants aged ≥40 years with VAS pain value ≥40 and <95 at |
| Osteoarthritis Pain (OA) | Visits 1 and 2 |
| | Presence of index knee pain for >12 weeks at Visit 1 |
| | Had an X-ray supporting diagnosis of OA according to the |
| | American College of Rheumatology with a Kellgren-Lawrence |
| | Grade 2 to 4 radiographic classification of index knee |
| H0P-MC-BP03 | Participants aged ≥18 years with VAS pain value ≥40 and <95 at |
| Chronic Low Back Pain (CLBP) | Visits 1 and 2 |
| | Had a history of daily pain for at least 12 weeks based on |
| | participant and report or medical history |
| | Had a history of lower back pain as classified by the Quebec |
| | TaskForce Category 1 through 3 |
| H0P-MC-NP03 | Participants aged ≥18 years with VAS pain value ≥40 and <95 at |
| Diabetic peripheral neuropathy | Visits 1 and 2 |
| (DPNP) | Had a history of daily pain for at least 12 weeks based on |
| | participant or medical history |
| | Had daily symmetrical foot pain secondary to peripheral |
| | neuropathy present for at least 6 months and as diagnosed |
| | through use of the Michigan Neuropathy Screening Instrument |
| | Part B ≥3 |

For each of these studies, further analyses were performed to identify TEAEs related to CV, thyroid, and abnormal renal function. CV AEs were identified using prespecified standardized MedDRA queries/preferred terms (SMQs/PTs) for cardiac arrhythmia, hypotension, major adverse cardiovascular event, and congestive heart failure. Thyroid-related AEs were identified with the ‘Hypothyroidism’ (SMQ), and AEs associated with abnormal renal function were identified with the high-level terms (HLTs) renal failure and impairment and select PTs.

Safety data from the 3 pooled intervention-specific appendix (ISAs), OA03, BP03, and NP03 were analyzed for the following safety topics of interest.

The pooled data from Studies OA03, BP03, and NP03 showed no clinically meaningful differences in thyroid-related TEAEs or thyroid analytes between Example 5- and placebo-treated patients.

Thus, after the treatment of a total 282 patients that were administered from 200 mg to 600 mg of Example 5 in the 3 phase 2 studies, an adequate safety profile was established. While the safety of the 3 phase 2 studies were discussed together, the efficacy results for each study will be discussed herein.

H0P-MC-OA03—Randomized, Placebo-Controlled, Phase 2 Clinical Trial to Evaluate Example 5 for the Treatment of Osteoarthritis Pain

The purpose of this study was to test whether Example 5 is efficacious in relieving osteoarthritis (OA) pain in the knee. Data was also collected to assess the safety and tolerability of Example 5 in this study population and previously discussed. Pharmacokinetic properties and pharmacodynamic effects were also be explored. The totality of data from this proof-of-concept study assessed the benefits and risks associated with Example 5.

OA03 was an 8-week, Phase 2, randomized, double-blind, placebo-controlled study that compared Example 5 versus placebo in participants with OA in the knee.

The primary clinical question of interest was “What is the treatment difference between Example 5 and placebo assessed using mean change from baseline to endpoint (Week 8) for average pain intensity (API) as measured using the numeric rating scale (NRS) in participants with OA of the knee regardless of initiation of rescue medication or other allowed concomitant medication and assuming that they would have continued initially randomized treatment condition?”

The key secondary clinical question of interest was: “What is the treatment difference between Example 5 and placebo assessed using mean change from baseline to endpoint (Week 8) for pain as measured by the WOMAC® (Western Ontario and McMaster Universities Arthritis Index) pain subscale in participants with OA of the knee regardless of initiation of rescue medication or other allowed concomitant medication and assuming that they would have continued initially randomized treatment condition?”

Participants were randomly assigned in the ratio of 2:1 to Example 5 or placebo. Based on tolerability, participants could take up to a maximum of 3 capsules orally (each capsule was 200 mg), twice daily (BID), approximately every 12 hours, for a total dose of 600 mg.

The protocol was amended to include a titration to the target dose of 600 mg. Investigators could reduce the dose from 600 to 400 mg after discussion with the participant due to tolerability as long as the change occurred before the end of Visit 4, that is, study Week 2. The medical monitor was contacted as needed.

A total of 202 participants enrolled into the OA03 ISA with 135 participants randomly assigned to Example 5 and 67 to placebo.

Demographic characteristics, disease history, and baseline characteristics were balanced across treatment groups, with an overall mean age of 62.8 years, a majority of females (57.4%), and an NRS rate average pain of 5.6 at baseline.

Duration of exposure (days) was calculated as the date of last dose in the treatment period minus the date of first dose plus 1. The mean number of days of exposure was 40.5 for the Example 5 group compared with 50.4 for the placebo group. Nine (6.7%) participants in the Example 5 group and 7 (10.6%) participants in the placebo group had at least 60 days of exposure. The protocol allowed changes to the dose for individual participants. 26 participants (13.0%) had “2 up-titrations and 1 down-titration” regimen: 21 participants (15.7%) were taking 200 mg, then increased to 400 mg, then increased to 600 mg, and then decreased to 400 mg BID of Example 5 and 5 participants (7.6%) were taking 1, then 2, then 3, and then decreased to 2 capsules BID of placebo.

The primary objective to evaluate the efficacy of Example 5 versus placebo on the overall mean change from baseline to endpoint (Week 8) for API as measured by weekly average of NRS did not meet the prespecified threshold for osteoarthritic pain.

The key secondary objective to evaluate the efficacy of Example 5 versus placebo on the overall mean change from baseline to endpoint (Week 8) for pain intensity measured by the WOMAC pain subscale did not meet the prespecified threshold.

Similarly, no statistical evidence that Example 5 is superior to placebo was observed for the other secondary endpoints in this study.

In total, in comparing Example 5 with placebo, there was no statistical evidence that Example 5 was superior to placebo for the primary, key secondary, or secondary endpoints in this study for the treatment of osteoarthritic pain (as shown in TABLE 5).

TABLE 5
Efficacy Summary of H0P-MC-OA03

| | | Placebo | Example 5 |
|---|---|---|---|
| Objective | Endpoint Measure | (n = 66) | (n = 135) |
| Primary | Mean change from baseline | −1.8 | −1.7 |
| Pain Intensity | assessment to endpoint (8 | | |
| Efficacy of | weeks after initial treatment | | |
| Example 5 versus | administration) for API as | | |
| Placebo | measured using the NRS | | |
| Key Secondary | Evaluate whether Example 5 is | −3.5 | −2.6 |
| Pain Intensity | superior to placebo in reducing | | |
| Efficacy of | pain as measured by the mean | | |
| Example 5 versus | change in pain intensity from | | |
| placebo | baseline assessment to Week 8 | | |
| | using the WOMAC ® pain | | |
| | subscale across all time points | | |

H0P-MC-BP03—Randomized, Placebo-Controlled, Phase 2 Clinical Trial to Evaluate Example 5 for the Treatment of Chronic Low Back Pain (CLBP)

The purpose of this study was to test whether Example 5 is efficacious in relieving chronic low back pain (CLBP). Data will be collected to assess the safety and tolerability of Example 5 in this study population, which was previously discussed. Pharmacokinetic (PK) properties and pharmacodynamic (PD) effects were also be explored. The totality of data from this proof-of-concept study will assess the benefits and risks associated with Example 5.

BP03 was an 8-week, Phase 2, randomized, double-blind, placebo-controlled study that will compare Example 5 versus placebo in participants with CLBP. Participants received either Example 5 or placebo. Based on tolerability, participants took up to a maximum of 3 capsules orally (each capsule is 200 mg), twice daily (BID), approximately every 12 hours, for a total dose of 600 mg.

The primary clinical question of interest was, “What is the treatment difference between Example 5 and placebo assessed using mean change from baseline to endpoint (Week 8) for average pain intensity (API) as measured by the numeric rating scale (NRS) in participants with CLBP regardless of initiation of rescue medication or other allowed concomitant medication and assuming that they would have continued initially randomized treatment condition?”

A total of 153 participants enrolled into the BP03 ISAs, with 102 participants randomly assigned to Example 5 and 51 participants randomly assigned to placebo.

Demographic characteristics, disease history, and baseline characteristics were balanced across treatment groups, with an overall mean age of 52.5 years, a majority of females (56.9%), and an NRS rate average pain of 5.7 at baseline.

Duration of exposure (days) was calculated as the date of last dose in the treatment period minus the date of first dose plus 1. The mean number of days of exposure was 47.5 for the Example 5 group compared with 50.8 for the placebo group. Seventeen (16.7%) participants in the Example 5 group and 7 (14.0%) participants in the placebo group had at least 60 days of exposure.

The protocol allowed changes to the dose for individual participants. Most participants (n=106; 69.7%) had “2 up-titrations” regimen: 61 participants (59.8%) were taking 200 mg, then increased to 400 mg, and then increased to 600 mg BID of Example 5, and 45 participants (90.0%) were taking 1, then 2, and then 3 capsules BID of placebo.

Thirty-five participants (23.0%) had “2 up-titrations and 1 down-titration” regimen: 32 participants (31.4%) were taking 200 mg, then increased to 400 mg, then increased to 600 mg, and then decreased to 400 mg BID of Example 5, and 3 participants (6.0%) were taking 1, then 2, then 3 capsules of placebo, and then decreased to 2 capsules BID of placebo.

The primary objective to evaluate the efficacy of Example 5 versus placebo on the overall mean change from baseline to endpoint (Week 8) for API as measured by weekly average of NRS did not meet the prespecified threshold. Similarly, no statistical evidence that Example 5 is superior to placebo was observed for the secondary endpoints in this study (as shown in TABLE 6).

In total, in comparing Example 5 with placebo, there was no statistical evidence that Example 5 was superior to placebo for the primary or secondary endpoints in this study for the treatment of chronic lower back pain (as shown in TABLE 6).

TABLE 6
Efficacy Summary of H0P-MC-BP03

| | | Placebo | Example 5 |
|---|---|---|---|
| Objective | Endpoint Measure | (n = 50) | (n = 102) |
| Primary | Mean change from baseline | −1.8 | −1.7 |
| Pain Intensity | assessment to endpoint (8 | | |
| Efficacy of | weeks after initial treatment | | |
| Example 5 versus | administration) for API as | | |
| Placebo | measured using the NRS | | |

H0P-MC-NP03—Randomized, Placebo-Controlled, Phase 2 Clinical Trial to Evaluate Example for the Treatment of Diabetic Peripheral Neuropathic Pain

The purpose of this study was to test whether Example 5 is efficacious in relieving diabetic peripheral neuropathic pain (DPNP). Data was collected to assess the safety and tolerability of Example 5 in this study population and previously discussed above. Pharmacokinetic properties and pharmacodynamic effects were also explored. The totality of data from this proof-of-concept study will assess the benefits and risks associated with Example 5.

NP03 was an 8-week, Phase 2, randomized, double-blind, placebo-controlled study that compared Example 5 versus placebo in participants with DPNP.

Participants received either Example 5 or placebo. Based on tolerability, participants received up to a maximum of 3 capsules orally (each capsule was 200 mg), twice daily, approximately every 12 hours, for a total dose of 600 mg.

The primary clinical question of interest was, “What is the treatment difference between Example 5 and placebo assessed using mean change from baseline to endpoint (Week 8) for average pain intensity (API) as measured by the Numeric Rating Scale (NRS) in participants with diabetic peripheral neuropathic pain (DPNP) regardless of initiation of rescue medication or other allowed concomitant medication and assuming that they would have continued initially randomized treatment condition?”

Participants were randomly assigned 2:1 to Example 5 or placebo. Based on tolerability, participants could take up to a maximum of 3 capsules orally (each capsule was 200 mg), twice daily (BID), approximately every 12 hours, for a total dose of 600 mg BID.

The protocol allowed changes to the dose for individual participants, but the maximum daily dose should not exceed 600 mg BID. The starting dose level was 200 mg BID. Decisions to change the dose level were made by the investigator in discussion with the participants based on the adverse events (AEs) reported for tolerability. The medical monitor was contacted as needed. All changes in dose level had to occur before the end of Visit 4 (Study Week 2).

The study enrolled men or women who met the following main criteria: had a visual analog scale (VAS) pain value 40 or more and less than 95 at Visits 1 and 2, had a history of daily pain for at least 12 weeks based on participant report or medical history, had a value of 30 or less on the Pain Catastrophizing Scale, had a body mass index of less than 40 kg/m2 (inclusive), had a daily symmetrical foot pain secondary to peripheral neuropathy present for at least 6 months and as diagnosed through use of the Michigan Neuropathy Screening Instrument Part B 3 or more, had a history and current diagnosis of type 1 or type 2 diabetes mellitus, and had a stable glycemic control as indicated by a glycated hemoglobin level of 11 or less at the time of screening.

Participants received either Example 5 or placebo. Based on tolerability, participants could take up to a maximum of 3 capsules orally (each capsule is 200 mg), BID, approximately every 12 hours, for a total dose of 600 mg.

The study included an 8-week DB treatment period.

A total of 68 participants enrolled into the NP03 intervention-specific appendix (ISA), with 45 participants randomly assigned to Example 5, and 23 participants randomly assigned to placebo. Demographic characteristics, disease history, and baseline characteristics were balanced across the treatment groups, with an overall mean age of 60.2 years for the Example 5 group compared to 58.3 years for the placebo group, a majority of females (55.9%), and a mean NRS rate average pain of 6.44 for the Example 5 group compared with 6.45 for the placebo group at baseline.

Duration of exposure (days) was calculated as the date of last visit in the treatment period minus the date of first dose+1. The mean number of days of exposure was 38.7 (min=4, max=64) for the Example 5 group compared with 45.7 (min=7, max=63) for the placebo group. Two (4.4%) participants in the Example 5 group and 3 (13.0%) participants in the placebo group had ≥60 days of exposure. Most participants (50, 73.5%) had “2 up-titrations” regimen. Eleven participants (16.2%) participants had “2 up-titrations and 1 down-titration” regimen.

The primary objective of this study was to evaluate the efficacy of Example 5 on the overall mean change from baseline to endpoint (Week 8) for API as measured by weekly average NRS (as shown in TABLE 7). The Example 5 group had a greater improvement in change from baseline in API compared with placebo at Week 8, and the posterior probability met the prespecified threshold. Similar findings were observed for bi-weekly average of API as measured by NRS.

Secondary objectives assessed pain severity and pain interference (Brief Pain Inventory-Short Form Modified [BPI-SFM]), overall improvement (Patient Global Impression of Change [PGIC]), worst pain intensity (NRS), pain intensity (VAS), sleep quality (Medical Outcomes Study Sleep Scale), and emotional functioning (European Quality of Life 5 Dimension 5 Level). For most secondary endpoints, greater numerical improvements were observed in the Example 5 group compared with placebo. The posterior probability met the prespecified threshold, indicating that Example 5 is superior to placebo, for the following endpoints:
mean change from baseline to endpoint for BPI-SFM individual severity scores:
‘at its worst in last 24 hours’ and ‘on the average’
mean change from baseline to endpoint for BPI-SFM individual inference scores:
‘with mood’ and ‘walking ability’
proportion of participants with reduction from baseline of at least 50% in BPI-SFM mean interference
proportion of participants with reduction from baseline of at least 30% in BPI-SFM individual interference scores:
‘with normal work’
overall improvement from baseline in PGIC
mean change from baseline in worst pain intensity—weekly average of NRS
mean change from baseline in worst pain intensity—biweekly average of NRS
time-to-first pain reduction of at least 50% in worst pain
mean change from baseline in VAS, and
proportion with at least 30% reduction in VAS score (Week 4 only).

NP03 was an 8-week, multicenter, randomized, DB, placebo-controlled, Phase 2, proof-of-concept study to evaluate Example 5 for the treatment of DPNP. Participants were randomly assigned 2:1 to Example 5 or placebo. A total of 68 participants enrolled into the NP03 ISA, with 45 participants randomly assigned to Example 5, and 23 participants assigned to placebo. The safety population consisted of the 68 participants who received at least 1 dose of study intervention and included all participants in the enrolled population. Using a titration scheme, Example 5 was administered orally as a maximum of 3 capsule (200 mg per capsule) for a total dose of 600 mg approximately every 12 hours, with or without food. Fifty percent of participants completed the BD treatment phase.

The primary objective of this study was to evaluate the efficacy of Example 5 on the overall mean change from baseline to endpoint (Week 8) for API as measured by weekly average NRS. The Example 5 group had a greater improvement in change from baseline in API compared with placebo at Week 8, and the posterior probability met the prespecified threshold. This demonstrates high confidence that Example 5, is superior to placebo relative to a standard of care-like effect at Week 8. Similar findings were observed for biweekly average of NRS.

TABLE 7 shows the proof-of-concept data to demonstrate efficacy of Example 5 in treating DPNP relative to placebo.

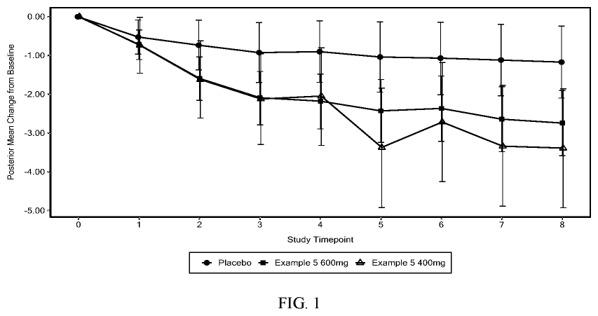

FIG. 1 shows the proof-of-concept data to demonstrate efficacy of Example 5 at 400 mg BID and 600 mg BID in treating DPNP relative to placebo.

TABLE 7
Efficacy Summary of H0P-MC-NP03

| | | Placebo | Example 5 |
|---|---|---|---|
| Objective | Endpoint Measure | (n = 23) | (n = 45) |
| Primary | Mean change from baseline | −1.19 | −2.75 |
| Pain Intensity | assessment to endpoint (8 weeks after | | |
| Efficacy of | initial treatment administration) for | | |
| Example 5 versus | API as measured using the NRS | | |
| Placebo | | | |

Secondary

| | | | |
|---|---|---|---|
| Physical | Mean change from baseline to | −1.27 | −2.31 |
| Functioning | endpoint for the BPI-SFM for the | | |
| Efficacy of | mean pain severity score | | |
| Example 5 | mean interference score | | |
| versus | individual severity score | | |
| Placebo | individual interference scores | | |
| Overall | Overall improvement to endpoint as | 3.2 | 2.3 |
| Improvement | measured by PGIC | | |
| Efficacy of | | | |
| Example 5 | | | |
| versus placebo | | | |
| Other Efficacy | Mean change from baseline | −1.06 | −3.06 |
| Efficacy of | assessment to endpoint for worst pain | | |
| Example 5 versus | intensity as measured by weekly | | |
| placebo | NRS | | |
| Other Efficacy | Mean change from baseline | −11.08 | −30.20 |
| Efficacy of | assessment to endpoint on the VAS | | |
| Example 5 versus | for pain | | |
| placebo | | | |

TABLE 8
Observed Mean of Average Pain Intensity as Measured by
Weekly Average of NRS by Dosing Regimens of Example 5

| Dose | Baseline | 1 | 2 | 3 | 4 | 5 | 6 | 7 | 8 |
|---|---|---|---|---|---|---|---|---|---|
| Placebo | 6.45 | 5.90 | 5.70 | 5.57 | 5.58 | 5.58 | 5.54 | 5.51 | 5.70 |
| | n = 23 | n = 23 | n = 21 | n = 18 | n = 18 | n = 16 | n = 16 | n = 15 | n = 14 |
| 400 mg | 6.47 | 5.76 | 4.90 | 4.41 | 5.46 | 4.66 | 4.15 | 2.96 | 4.17 |
| BID | n = 9 | n = 8 | n = 9 | n = 9 | n = 6 | n = 4 | n = 5 | n = 4 | n = 4 |
| 600 mg | 6.45 | 5.77 | 4.91 | 4.82 | 4.64 | 4.18 | 4.26 | 3.78 | 3.79 |
| BID | n = 31 | n = 30 | n = 29 | n = 23 | n = 24 | n = 21 | n = 19 | n = 17 | n = 16 |

TABLE 9
Mean Change of Average Pain Intensity as Measured by Weekly
Average of NRS by Dosing Regimens of Example 5

| Dose | Baseline | 1 | 2 | 3 | 4 | 5 | 6 | 7 | 8 |
|---|---|---|---|---|---|---|---|---|---|
| Placebo | 6.45 | −0.52 | −0.73 | −0.93 | −0.90 | −1.04 | −1.07 | −1.12 | −1.17 |
| | n = 23 | n = 23 | n = 21 | n = 18 | n = 18 | n = 16 | n = 16 | n = 15 | n = 14 |
| 400 mg | 6.47 | −0.74 | −1.62 | −2.12 | −2.05 | −3.37 | −2.72 | −3.34 | −3.39 |
| BID | n = 9 | n = 8 | n = 9 | n = 9 | n = 6 | n = 4 | n = 5 | n = 4 | n = 4 |
| 600 mg | 6.45 | −0.72 | −1.59 | −2.09 | −2.18 | −2.43 | −2.37 | −2.64 | −2.74 |
| BID | n = 31 | n = 30 | n = 29 | n = 23 | n = 24 | n = 21 | n = 19 | n = 17 | n = 16 |

The efficacy of Example 5 on the overall mean change from baseline to endpoint (Week 8) for API as measured by weekly average NRS by dosing regimen using the Bayesian repeated measures analysis with PS method is presented in TABLEs 8 and 9 and FIG. 1. At Week 8, there was a greater decrease in API measured by NRS in the 400 mg dosing regimen compared with the 600 mg dosing regimen.

Surprisingly, clinical efficacy was demonstrated in diabetic peripheral neuropathy, but not in chronic lower back pain and osteoarthritis of the knee, despite similar doses being studied. Additionally, it is believed that this efficacy results are the solution to a long-felt need of finding a non-opioid pain treatment without severe side effects.

### Example 9

Protocol for a Phase II(b) Dose Regimen study in DPNP (Study J2P-MC-LXBD)

Disclosed herein is also a protocol for a dose regimen study in DPNP with certain doses of the dosing regimens of the present invention. The skilled artisan will be able to apply the teachings of this Example 8 and other disclosures provided herein and conduct similar studies with additional doses and dosing regimens of the present invention.

The purpose of this study will be to test whether Example 5 is efficacious in relieving diabetic peripheral neuropathic pain (DPNP) at additional doses.

LXBD will be a 12 week, Phase 2, randomized, double-blind placebo-controlled study that compares Example 5 versus placebo in participants with DPNP.

Participants will receive either Example 5 or placebo. Patients will be split into 4 randomized groups in a 2:1:1:1 ratio for placebo:treatment group 1:treatment group 2:treatment group 3. Each randomized group will be allowed 4 weeks to achieve full titration in all arms.

Based on tolerability, participants in treatment group 1 will receive up to a maximum of 3 capsules orally (each capsule including 50 mg of Example 5 or placebo), twice daily, approximately every 12 hours, for a total dose of 50 mg.

Based on tolerability, participants in treatment group 1 will receive up to a maximum of 3 capsules orally (each capsule including 50 mg of Example 5 or placebo), twice daily, approximately every 12 hours, for a total dose of 50 mg. There is no currently projected titration schedule for treatment group 1, thus, the participants will receive 50 mg of Example 5 for 12 weeks.

Based on tolerability, participants in treatment group 2 will receive up to a maximum of 3 capsules orally (each capsule including 50 mg of Example 5, 200 mg of Example 5, or placebo), twice daily, approximately every 12 hours, for a total dose of 200 mg of Example 5. The currently projected titration schedule for treatment group 2, will include week 1 at 50 mg BID, week 2 at 100 mg BID, and weeks 3-12 at 200 mg BID subject to tolerability of higher doses of Example 5 for participants.

Based on tolerability, participants in treatment group 3 will receive up to a maximum of 3 capsules orally (each capsule including 50 mg of Example 5, 200 mg of Example 5, or placebo), twice daily, approximately every 12 hours, for a total dose of 400 mg of Example 5. The currently projected titration schedule for treatment group 2, will include week 1 at 50 mg BID, week 2 at 100 mg BID, week 3 at 200 mg BID, week 4 at 300 mg BID, and weeks 5-12 at 400 mg BID subject to tolerability of higher doses of Example 5 for participants.

Participants may be allowed move from treatment group 3 to treatment group, from treatment group 2 to treatment group 1, or from treatment group 3 to treatment group 1 based on tolerability of the intended dose to minimize drop out of the patients in the study. LXBD will provide safety information (TEAE's frequency, severity, duration, DCAE) for each of the dose arms and for each of the titration steps.

In total, three treatment doses will be evaluated to meet primary efficacy endpoints: 50 mg, 200 mg, and 400 mg BID (twice daily).

What is claimed is:

1. A method of treating pain in a patient in need of treatment thereof, the method comprising administering to the patient a dose of about 25 mg to about 1400 mg per dose of a compound of the formula: 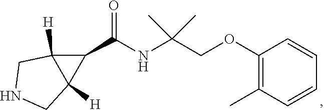 [SMILES: Cc1ccccc1OCC(C)(C)NC(=O)[C@H]1[C@@H]2CNC[C@@H]21]
or a pharmaceutically acceptable salt thereof, and/or a hydrate thereof.

2. The method of claim 1, wherein the compound is of the formula: 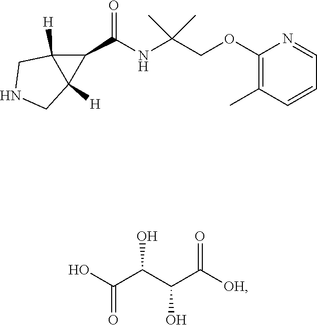 [SMILES: Cc1cccnc1OCC(C)(C)NC(=O)[C@H]1[C@@H]2CNC[C@@H]21 ; O=C(O)[C@H](O)[C@@H](O)C(=O)O]
or a hydrate thereof.

3. The method of claim 2, wherein the compound is of the formula:

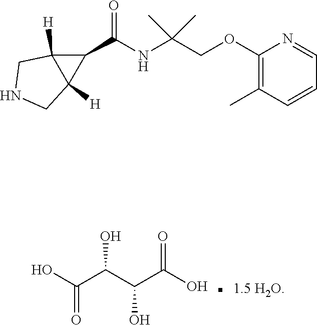 [SMILES: Cc1cccnc1OCC(C)(C)NC(=O)[C@H]1[C@@H]2CNC[C@@H]21 ; O=C(O)[C@H](O)[C@@H](O)C(=O)O]

4. The method of claim 2, wherein the compound is of the formula:

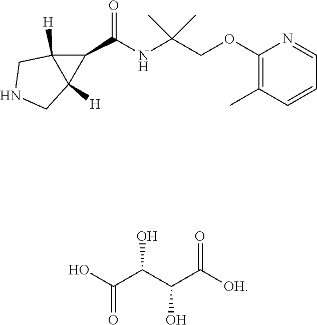 [SMILES: Cc1cccnc1OCC(C)(C)NC(=O)[C@H]1[C@@H]2CNC[C@@H]21 ; O=C(O)[C@H](O)[C@@H](O)C(=O)O]

5. The method of claim 1, wherein the dose is administered to the patient once daily, twice daily, every 24 hours, every 12 hours, every 8 hours, every 6 hours, or every 4 hours.

6. The method of claim 5, wherein the dose is administered to the patient twice daily.

7. The method of claim 1, wherein the dose administered to the patient is from about 50 mg to about 600 mg.

8. The method of claim 7, wherein the dose administered to the patient is about 50 mg, about 100 mg, about 200 mg, about 300 mg, about 400 mg, about 500 mg, or about 600 mg.

9. The method of claim 8, wherein the dose administered to the patient is about 50 mg and the dose is administered twice daily.

10. The method of claim 8, wherein the dose administered to the patient is about 100 mg and the dose is administered twice daily.

11. The method of claim 8, wherein the dose administered to the patient is about 200 mg and the dose is administered twice daily.

12. The method of claim 8, wherein the dose administered to the patient is about 300 mg and the dose is administered twice daily.

13. The method of claim 8, wherein the dose administered to the patient is about 400 mg and the dose is administered twice daily.

14. The method of claim 8, wherein the dose administered to the patient is about 500 mg and the dose is administered twice daily.

15. The method of claim 8, wherein the dose administered to the patient is about 600 mg and the dose is administered twice daily.

16. The method of claim 1, wherein the pain comprises osteoarthritic pain, chronic lower back pain, or neuropathic pain.

17. The method of claim 1, wherein the pain comprises neuropathic pain.

18. The method of claim 17, wherein the neuropathic pain comprises diabetic neuropathy, polyneuropathy, distal sensory polyneuropathy, peripheral neuropathy, central neuropathy, diabetic peripheral neuropathy, or mixed neuropathy.

19. The method of claim 1, wherein the compound is administered to the patient in a tablet composition, a capsule composition, or an aqueous solution.

20. The method of claim 19, wherein the capsule composition comprises microcrystalline cellulose, silica dioxide, or combination thereof.

21. A capsule composition comprising a compound of the formula: 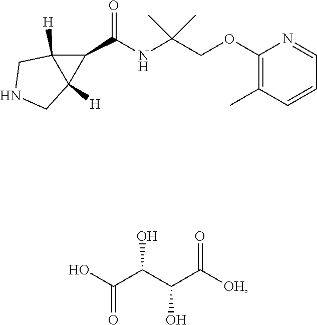 [SMILES: Cc1cccnc1OCC(C)(C)NC(=O)[C@H]1[C@@H]2CNC[C@@H]21 ; O=C(O)[C@H](O)[C@@H](O)C(=O)O]
or a hydrate thereof.

22. The composition of claim 21, wherein the compound is of the formula:

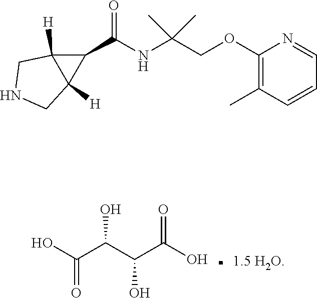 [SMILES: Cc1cccnc1OCC(C)(C)NC(=O)[C@H]1[C@@H]2CNC[C@@H]21 ; O=C(O)[C@H](O)[C@@H](O)C(=O)O]

23. The composition of claim 21, wherein the capsule composition further comprises microcrystalline cellulose, silica dioxide, or combination thereof.

24. The composition of claim 21, wherein the composition comprises from about 50 mg to about 600 mg of the compound.

25. The composition of claim 21, wherein the composition comprises from about 50 mg to about 200 mg of the compound.

26. The composition of claim 21, wherein the composition comprises about 50 mg of the compound.

27. The composition of claim 21, wherein the composition comprises about 200 mg of the compound.

28. A method for preparing a compound of the formula: 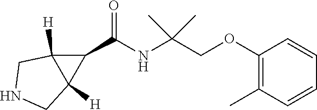 [SMILES: Cc1ccccc1OCC(C)(C)NC(=O)[C@H]1[C@@H]2CNC[C@@H]21]
or pharmaceutically acceptable salts thereof, and
the method comprising:
mixing a compound of the formula: 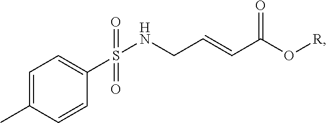
wherein R is a C₁ to C₆ alkyl,
with a sulfonium salt of the formula: 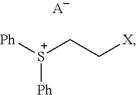
wherein X is a halogen and A is an anion to yield an intermediate compound of the formula: 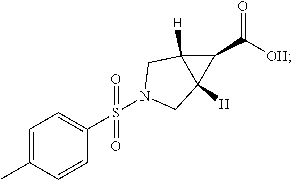 [SMILES: Cc1ccc(S(=O)(=O)N2C[C@@H]3[C@H](C2)[C@H]3C(=O)O)cc1]
mixing the intermediate compound with 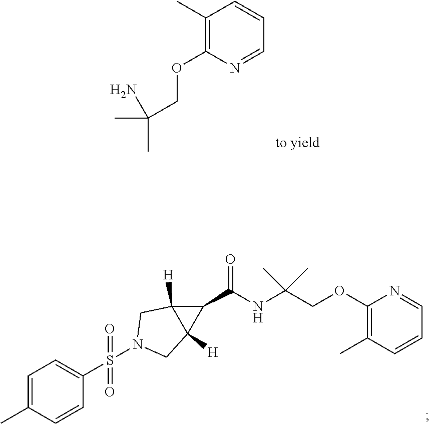 [SMILES: Cc1cccnc1OCC(C)(C)N ; Cc1ccc(S(=O)(=O)N2C[C@@H]3[C@H](C2)[C@H]3C(=O)NC(C)(C)COc2ncccc2C)cc1]
and
removing tosyl function group from 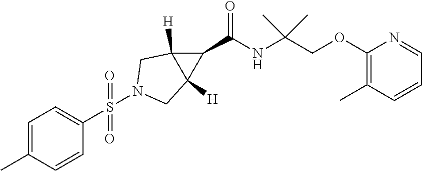 [SMILES: Cc1ccc(S(=O)(=O)N2C[C@@H]3[C@H](C2)[C@H]3C(=O)NC(C)(C)COc2ncccc2C)cc1]
to yield the compound.

29. The method of claim 28, wherein the method comprises:
mixing a compound of the formula: 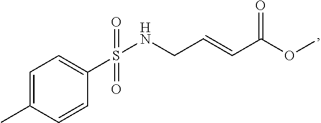
with a sulfonium salt of the formula: 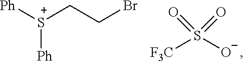 [SMILES: BrCC[S+](c1ccccc1)c1ccccc1 ; O=S(=O)([O-])C(F)(F)F]
to yield the intermediate compound.

30. The method of claim 28, wherein the method comprises less than 8 synthetic steps.

31. The method of claim 28, wherein the method does not include a metal catalyst.

32. The method of claim 28, wherein the method does not utilize a hydride salt.

33. The method of claim 28, wherein the method comprises:
(a) mixing 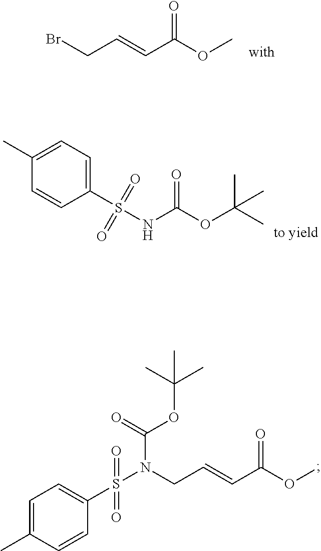
(b) mixing 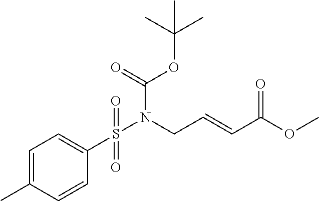
with trifluoroacetic acid to yield 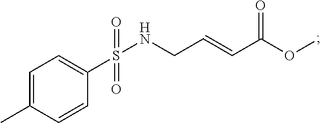
and
(c) mixing 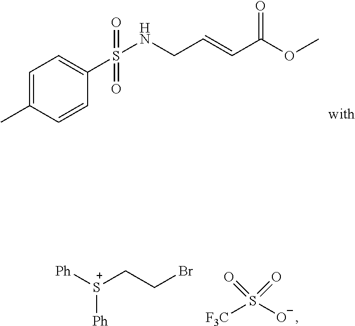
to yield the intermediate compound.

34. The method of claim 33, wherein the method further comprises:
(d) mixing the intermediate compound with 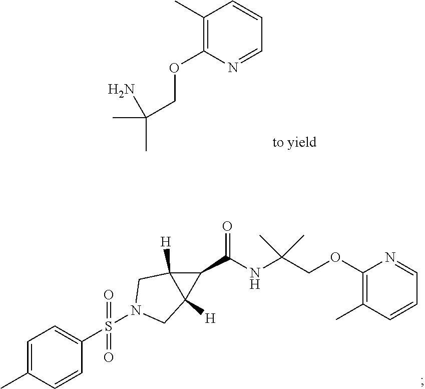 [SMILES: Cc1cccnc1OCC(C)(C)N ; Cc1ccc(S(=O)(=O)N2C[C@@H]3[C@H](C2)[C@H]3C(=O)NC(C)(C)COc2ncccc2C)cc1]
and
(e) mixing 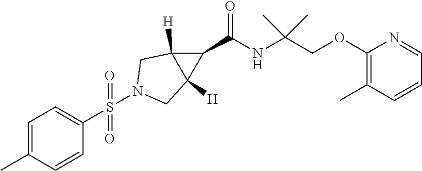 [SMILES: Cc1ccc(S(=O)(=O)N2C[C@@H]3[C@H](C2)[C@H]3C(=O)NC(C)(C)COc2ncccc2C)cc1]
with potassium diphenylphosphine to yield the compound, or a pharmaceutically acceptable salt thereof and/or a hydrate thereof.

35. The method of claim 28, wherein the method comprises:
(a) mixing methyl (E) 4-bromobut-2-enoate with tert-Butyl tosylcarbamate to yield methyl (E)-4-((N-(tert-butoxycarbonyl)-4-methylphenyl)sulfonamido)but-2-enoate;
(b) mixing methyl (E)-4-((N-(tert-butoxycarbonyl)-4-methylphenyl)sulfonamido)but-2-enoate with trifluoroacetic acid to yield methyl (E)-4-((4-methylphenyl)sulfonamido)but-2-enoate;
(c) mixing methyl (E)-4-((4-methylphenyl)sulfonamido)but-2-enoate with 2-bromoethyl)diphenylsulfonium triflate to yield the intermediate compound;
(d) mixing the intermediate compound with 2-Methyl-1-((3-methylpyridin-2-yl)oxy)propan-2-amine to yield (1R,5S,6r)-N-(2-Methyl-1-((3-methylpyridin-2-yl)oxy)propan-2-yl)-3-tosyl-3-azabicyclo[3.1.3]hexane-6-carboxamide;
(e) mixing ((1R,5S,6r)-N-(2-Methyl-1-((3-methylpyridin-2-yl)oxy)propan-2-yl)-3-tosyl-3-azabicyclo[3.1.3]hexane-6-carboxamide with potassium diphenylphosphine to yield the compound, or a pharmaceutically acceptable salt thereof and/or a hydrate thereof.

36. The method of claim 28, wherein the compound is selected from 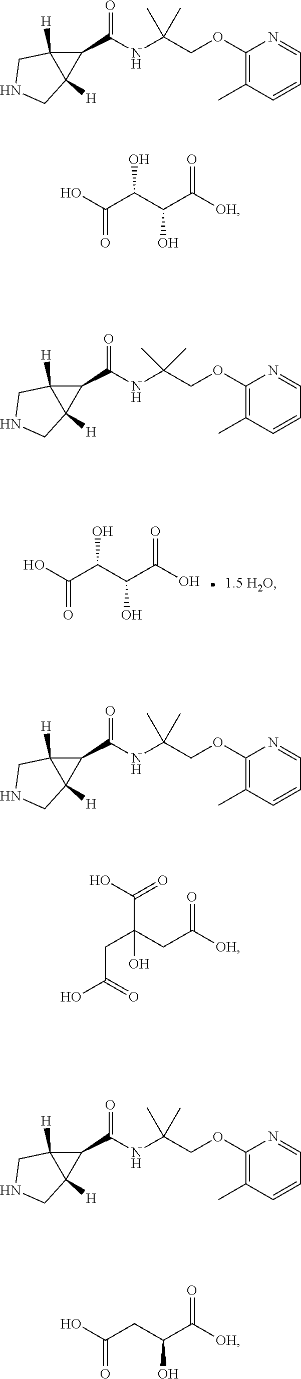
or combinations thereof.

37. The method of claim 24, wherein the method further comprises:
(f) mixing the compound with L-tartaric acid, citric acid, L-malic acid, or a combination thereof.

38. A method for preparing an intermediate compound of the formula: 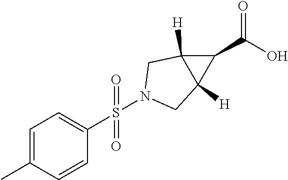 [SMILES: Cc1ccc(S(=O)(=O)N2C[C@@H]3[C@H](C2)[C@H]3C(=O)O)cc1]
or pharmaceutically acceptable salts thereof, and
the method comprising:
mixing a compound of the formula: 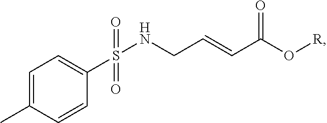
wherein R is a C₁ to C₆ alkyl,
with a sulfonium salt of the formula: 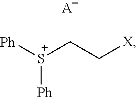
wherein X is a halogen and A is an anion to yield the intermediate compound.

39. The method of claim 38, wherein the method comprises:
mixing a compound of the formula: 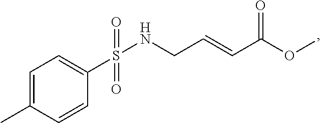
with a sulfonium salt of the formula: 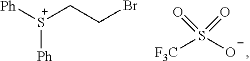 [SMILES: BrCC[S+](c1ccccc1)c1ccccc1 ; O=S(=O)([O-])C(F)(F)F]
to yield the intermediate compound, or a pharmaceutically acceptable salt thereof.

40. The method of claim 38, wherein the method comprises less than 8 synthetic steps.

41. The method of claim 38, wherein the method does not include a metal catalyst.

42. The method of claim 38, wherein the method does not utilize a hydride salt.

43. The method of any one of claims 38 to 42, wherein the method:
(a) mixing 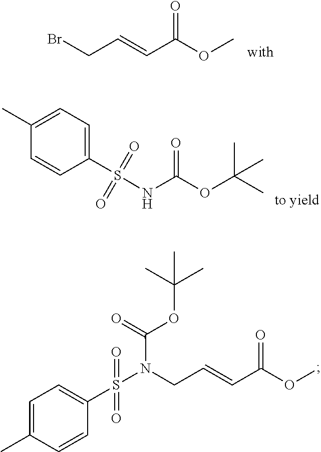
(b) mixing 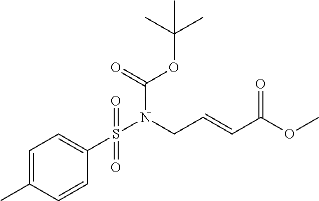
with trifluoroacetic acid to yield 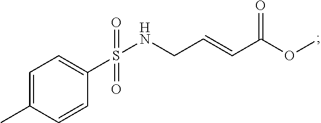
and
(c) mixing 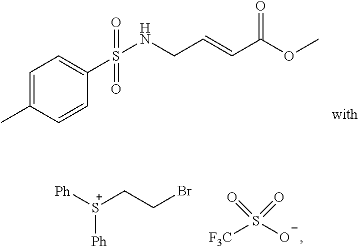
to yield the intermediate compound, or a pharmaceutically acceptable salt thereof.

44. The method of claim 38, wherein the method comprises:
(a) Mixing methyl (E) 4-bromobut-2-enoate with tert-Butyl tosylcarbamate to yield methyl (E)-4-((N-(tert-butoxycarbonyl)-4-methylphenyl)sulfonamido)but-2-enoate;
(b) Mixing methyl (E)-4-((N-(tert-butoxycarbonyl)-4-methylphenyl)sulfonamido)but-2-enoate with trifluoroacetic acid to yield methyl (E)-4-((4-methylphenyl)sulfonamido)but-2-enoate; and
(c) Mixing methyl (E)-4-((4-methylphenyl)sulfonamido)but-2-enoate with 2-bromoethyl)diphenylsulfonium triflate to yield the intermediate compound, or a pharmaceutically acceptable salt thereof.

45. A compound of the formula: 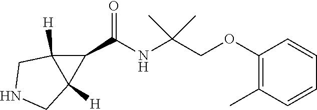 [SMILES: Cc1ccccc1OCC(C)(C)NC(=O)[C@H]1[C@@H]2CNC[C@@H]21]
or a pharmaceutically acceptable salts thereof and/or a hydrate thereof,
wherein the compound is prepared by the method of claim 28.

46. The compound of claim 45, wherein the compound is of the formula: 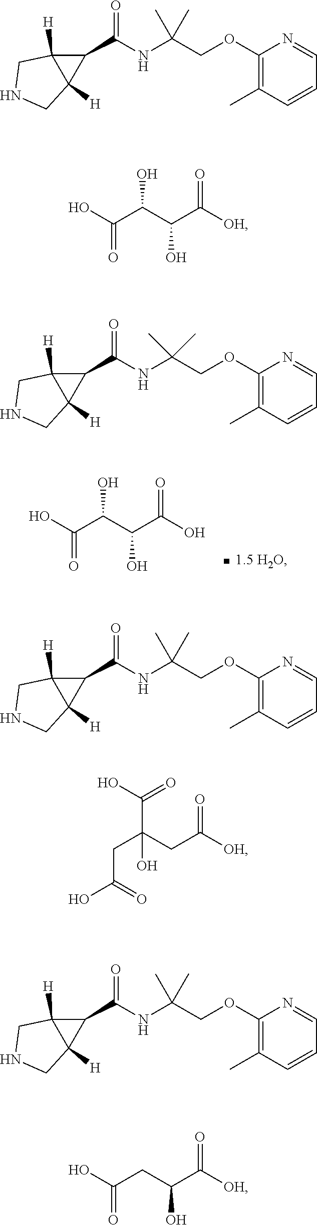
or combinations thereof.

47. A compound prepared by the method of claim 28, wherein the compound is of the formula: 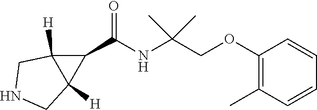 [SMILES: Cc1ccccc1OCC(C)(C)NC(=O)[C@H]1[C@@H]2CNC[C@@H]21]
or a pharmaceutically acceptable salt thereof and/or a hydrate thereof.

47. A compound prepared by the method of claim 28, wherein the compound is of the formula: 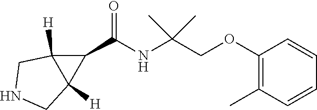 [SMILES: Cc1ccccc1OCC(C)(C)NC(=O)[C@H]1[C@@H]2CNC[C@@H]21]
or a pharmaceutically acceptable salt thereof and/or a hydrate thereof.

48. The compound of claim 47, wherein the compound is selected from: 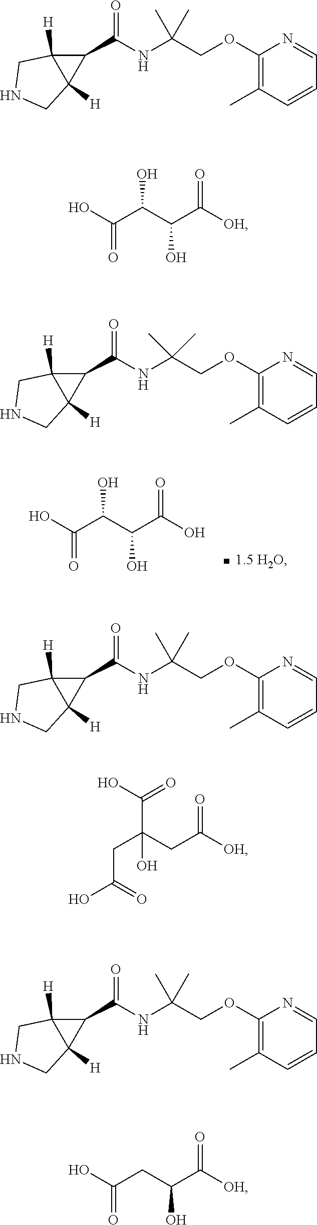
or combinations thereof.

49-68. (canceled)
